(19) United States
(12) Patent Application Publication
(10) Pub. No.: US 2025/0391501 A1
(43) Pub. Date: Dec. 25, 2025

(54) INTEGRATED FRAMEWORK FOR HUMAN EMBRYO PLOIDY PREDICTION USING ARTIFICIAL INTELLIGENCE

(71) Applicant: Cornell University, Ithaca, NY (US)

(72) Inventors: Iman HAJIRASOULIHA, New York, NY (US); Nikica ZANINOVIC, New York, NY (US); Josue BARNES, New York, NY (US); Zev ROSENWAKS, New York, NY (US); Olivier ELEMENTO, New York, NY (US); Jonas MALMSTEN, Gibsonton, FL (US)

(73) Assignee: Cornell University, Ithaca, NY (US)

(21) Appl. No.: 18/834,931

(22) PCT Filed: Feb. 10, 2023
(86) PCT No.: PCT/US2023/062368

§ 371 Date: Jul. 31, 2024

Related U.S. Application Data
(60) Provisional application No. 63/308,710, filed on Feb. 10, 2022.
(60) Provisional application No. 63/433,197, filed on Dec. 16, 2022.

(51) Int. Cl. G16B 20/10; G06T 7/00; G16B 40/20; G16H 30/40

(52) U.S. Cl. CPC G16B 20/10; G06T 7/0012; G16B 40/20; G16H 30/40; G06T 2207/10056; G06T 2207/20081; G06T 2207/30044

(57) ABSTRACT

The present disclosure encompasses systems and methods for predicting embryo ploidy. Specific embodiments encompass methods of non-invasively predicting ploidy status of an embryo, by receiving a dataset with a static image of the embryo, analyzing the static image by one or more machine and/or deep learning model via one or more classification task applied to the dataset; and generating an output prediction of the ploidy status of the embryo. Particular methods relate to methods wherein the dataset additionally includes one or more clinical and/or morphological features for the embryo. Embodiments also relate to predicting embryo viability and/or improving embryo selection, such as during in vitro fertilization, and uses thereof.

### CROSS REFERENCE TO RELATED APPLICATIONS

The present application claims the benefit of priority under 35 U.S.C. § 119(e) to U.S. Provisional Application No. 63/308,710, INTEGRATED FRAMEWORK FOR HUMAN EMBRYO PLOIDY PREDICTION USING ARTIFICIAL INTELLIGENCE, filed on Feb. 10, 2022; and U.S. Provisional Application No. 63/433,197, INTEGRATED FRAMEWORK FOR HUMAN EMBRYO PLOIDY PREDICTION USING ARTIFICIAL INTELLIGENCE, filed on Dec. 16, 2022; each of which are incorporated by reference in their entirety.

### STATEMENT REGARDING FEDERALLY-SPONSORED RESEARCH

This invention was made with government support under Grant Nos. R35 GM138152-01 and TL1-TR-002386 awarded by the National Institutes of Health. The government has certain rights in the invention.

### FIELD

The present disclosure relates generally to the field of assisted reproduction, and particularly relates to systems, software, and methods for evaluating embryos by, for example, predicting embryo ploidy status.

### BACKGROUND

A challenge in the field of in vitro fertilization (IVF) is the selection of the most viable embryos for transfer. Current methods of embryo selection include morphological quality assessment and morphokinetic analysis; however, both suffer from intra- and inter-observer variability, particularly due to the unstandardized methods. A third method, pre-implantation genetic testing for aneuploid (PGT-A) also has notable limitations, including its invasiveness and cost.

Current methods of embryo selection for transfer during IVF suffer from inter- and intra-observer bias as observed in morphological assessment and morphokinetic annotation or present an ethical barrier as seen in invasive trophectoderm biopsies for PGT-A. Several recent studies have sought to alleviate the limitation of morphological assessment by utilizing deep learning to predict embryo quality. However, fewer studies, have sought to use deep learning to predict embryo ploidy as a standardized method of embryo selection.

Differences in aneuploid and euploid embryos that allow for model-based classification are reflected in morphology, morphokinetics, and associated clinical information. As such, there is a need in the industry to provide automated, non-invasive tools for evaluating and selecting embryos for transfer during IVF via model-based classification.

### SUMMARY OF THE INVENTION

Various embodiments of the invention relate to non-invasive methods of predicting ploidy status of an embryo, the method including: receiving a dataset comprising a static image of the embryo; analyzing the dataset by one or more machine and/or deep learning model via one or more classification task applied to the dataset; and generating an output prediction of the ploidy status of the embryo. Some embodiments of the methods further include acquiring the static image. Some embodiments of the methods further includes training the one or more machine learning model using training data, where the training data includes a plurality of probabilities, and/or model- or embryologist-derived or provided clinical features for a plurality of subjects and a plurality of embryo ploidy statuses for the plurality of subjects. In some embodiments, the method is automated.

Embodiments of the invention also relate to user interfaces for predicting ploidy status of an embryo, the user interface including: a web-based platform for uploading and analyzing a dataset, wherein the dataset includes a static image of the embryo; analysis software integrated with the web-based platform to analyze the dataset by one or more machine and/or deep learning model via one or more classification task applied to the dataset; and an output generation which provides a prediction of ploidy status of the embryo.

In some embodiments, the prediction of the ploidy status of the embryo includes a probability. In some embodiments, the probability includes a probability of the embryo being euploid. In some embodiments, the classification task can be a binary classification task. In some embodiments, the binary classification task provides a probability for the embryo of being aneuploid vs. euploid; complex aneuploid vs. euploid or single aneuploid; or complex aneuploid vs. euploid. In some embodiments, the binary classification task provides a probability for the embryo of being aneuploid vs. euploid. In some embodiments, the binary classification task provides a probability for the embryo of being complex aneuploid vs. euploid or single aneuploid. In some embodiments, the binary classification task provides a probability for the embryo of being complex aneuploid vs. euploid.

In some embodiments, the static image can be acquired via time-lapse microscopy. In some embodiments, the static image can be captured at Day 5 or Day 6 of embryo development. In some embodiments, the static image can be captured from 105-115, or from 109-111 hours post insemination (hpi). In some embodiments, the static image can be captured at or about 110 hours post insemination (hpi). In some embodiments, one individual static image can be captured and analyzed per embryo.

In some embodiments, the dataset further includes one or more clinical and/or morphological features for the embryo. In some embodiments, the clinical and/or morphological features include one or more morphokinetic parameters/annotations, one or more blastocyst morphological assessments, maternal age at the time of oocyte retrieval, and/or preimplantation genetic testing for aneuploidy (PGT-A).

In some embodiments, the blastocyst morphological assessments include blastocyst grade (BG), blastocyst score (BS), and/or artificial intelligence-driven predicted blastocyst score (AIBS). In some embodiments, BS can be determined based on machine and/or deep learning and regression analysis. In some embodiments, the BS score determination includes converting inner cell mass (ICM), trophectoderm (TE), and/or expansion grades into numerical values. In some embodiments, the BS score determination includes converting inner cell mass (ICM), trophectoderm (TE), and/or expansion grades into numerical values, and additionally includes an input based on day of blastocyst formation. In some embodiments, BS can include a numerical value based on ICM, TE, and expansion grade. In some embodiments, BS can further include a score based on day of blastocyst formation.

In some embodiments, the morphokinetic parameters comprise time of pro-nuclear fading (tPnF), time to 2 cells (t2), time to 3 cells (t3), time to 4 cells (t4), time to 5 cells (t5), time to 6 cells (t6), time to 7 cells (t7), time to 8 cells (t8), time to 9 cells (t9), time of morula (tM), and/or time of the start of blastulation (tSB). In some embodiments, analyzing morphokinetic parameters includes assigning blastocyst grade (BG) using a grading system. In some embodiments, the grading system includes assessments of inner cell mass (ICM), trophectoderm (TE), and/or expansion.

In some embodiments, the clinical features include maternal age and/or blastocyst score (BS). In some embodiments, maternal age and/or blastocyst score (BS) can be weighted more heavily than other clinical features based on one or more classification task. In some embodiments, TE score can be weighted more heavily than other blastocyst score factors based on one or more classification tasks.

In some embodiments, the clinical and/or morphological features include one or more of maternal age at the time of oocyte retrieval, blastocyst grade-inner cell mass, blastocyst grade-trophectoderm, blastocyst grade-expansion, blastocyst score, time of pro-nuclear fading (tPnF), time to 2 cells (t2), time to 3 cells (t3), time to 4 cells (t4), time to 5 cells (t5), time to 6 cells (t6), time to 7 cells (t7), time to 8 cells (t8), time to 9 cells (t9), time of morula (tM), and/or time of the start of blastulation (tSB). In some embodiments, the clinical and/or morphological features can be weighted in order of maternal age at the time of oocyte retrieval, blastocyst, blastocyst score, and/or morphokinetic parameters. In some embodiments, blastocyst score can correlate positively, and/or maternal age caxn correlate negatively with embryo ploidy. In some embodiments, the maternal age can be 37 or younger, and the embryo can have a higher probability of being euploid.

In some embodiments, the dataset can be pre-processed prior to analysis. In some embodiments, pre-processing the dataset includes removing faulty static images and/or imputing values for any missing morphokinetic parameters via median imputation. In some embodiments, a faulty image includes an image that cannot be processed and/or analyzed. In some embodiments, an image that cannot be processed and/or analyzed can be over- or under-exposed. In some embodiments, the dataset includes values for each morphokinetic parameter following pre-processing.

In some embodiments, the analysis includes regression analysis. In some embodiments, the regression analysis includes a LASSO regression and/or logistic regression applied to one or more clinical features. In some embodiments, the regression analysis can be used to weight importance of one or more clinical features. In some embodiments, the analysis includes determination of an artificial intelligence-driven predicted blastocyst score (AIBS) for the embryo.

In some embodiments, the static image(s) and clinical features can be combined and analyzed by machine and/or deep learning in two fully-connected layers. In some embodiments, the analysis can output a predicted embryo ploidy in a binary classification task. In some embodiments, the machine learning includes a convolutional neural network (CNN). In some embodiments, the machine learning includes a ResNet18 CNN architecture. In some embodiments, the machine learning includes Extreme Gradient Boost Decision Tree (XGBoost), k-nearest neighbor (k-NN), support vector machine (SVM), and/or Random Forest.

In some embodiments, a prediction of embryo ploidy status can be used to predict embryo viability, wherein an embryo having a stronger probability of being euploid has a higher probability of being viable. In some embodiments, a prediction of embryo ploidy status can be used to improve embryo selection for implantation during in vitro fertilization, wherein an embryo having a stronger probability of being euploid can be selected. In some embodiments, a prediction of embryo ploidy status can be used for selecting and/or prioritizing an embryo for preimplantation genetic testing for aneuploidy (PGT-A) biopsy and/or implantation during in vitro fertilization. In some embodiments, a prediction of embryo ploidy status can be used in combination with traditional methods of embryo selection and prioritization for implantation and/or recommendation for PGT-A during in vitro fertilization. In some embodiments, a prediction of embryo ploidy status can be used to improve an outcome in a subject undergoing in vitro fertilization, wherein an embryo predicted to be euploid can be selected for embryo transfer during in vitro fertilization.

Embodiments of the invention also relate to systems including one or more data processors; and a non-transitory computer readable storage medium containing instructions which, when executed on the one or more data processors, cause the one or more data processors to perform part or all of one or more of the following steps: acquiring a static image of an embryo; receiving a dataset comprising the static image of the embryo; analyzing the dataset by one or more machine and/or deep learning model via one or more classification task applied to the dataset; generating an output prediction of the ploidy status of the embryo; training the one or more machine learning model using training data, where the training data includes a plurality of probabilities, and/or model- or embryologist-derived or provided clinical features for a plurality of subjects and a plurality of embryo ploidy statuses for the plurality of subjects. In some embodiments, the execution is automated.

Embodiments of the invention also relate to computer-program products tangibly embodied in a non-transitory machine-readable storage medium, including instructions configured to cause one or more data processors to perform part or all of one or more of the following steps: acquiring a static image of an embryo; receiving a dataset comprising the static image of the embryo; analyzing the dataset by one or more machine and/or deep learning model via one or more classification task applied to the dataset; generating an output prediction of the ploidy status of the embryo; training the one or more machine learning model using training data, where the training data includes a plurality of probabilities, and/or model- or embryologist-derived or provided clinical features for a plurality of subjects and a plurality of embryo ploidy statuses for the plurality of subjects. In some embodiments, the performance is automated.

### BRIEF DESCRIPTION OF THE DRAWINGS

Those of skill in the art will understand that the drawings, described below, are for illustrative purposes only. The drawings are not intended to limit the scope of the present teachings in any way.

FIG. 1. An example computer system, upon which embodiments, or portions of the embodiments, may be implemented, in accordance with various embodiments.

FIG. 2. Study design and exemplary STORK-A schematic workflow, in accordance with various embodiments. FIG. 2A) Time-lapse videos are extracted from the Embryoscope®, and a single static image at 110 hours post insemination (hpi) (focal plane 0) is used for each embryo, along with morphokinetic annotations, morphological assessments, maternal age, and/or associated PGT-A results. The dataset is then pre-processed to remove underexposed images by manual detection, and missing morphokinetic values are imputed using median imputation. LASSO and logistic regressions are then applied to clinical information to determine feature importance. After determining feature importance, the dataset is split 70/15/15 for training, validating, and testing models to predict embryo ploidy. Hyperparameters for the models are then optimized through iterative training and once completed, the performance on the test set is evaluated. FIG. 2B) Exemplary overview of the deep learning model for ploidy classification. Image features extracted from the ResNet18 CNN are concatenated with clinical information (maternal age, morphokinetic parameters, and one of three morphological assessments BG, BS, AIBS) before being passed on to a final fully-connected layer.

FIG. 3. Clinical feature importance example for complex aneuploid vs. euploid prediction. The x-axis indicates the degree of importance of each of the clinical information features, or the mean weight associated with LASSO regression using five-fold cross-validation (error bars represent 95% CI).

FIG. 4. Determination of weights for each feature and validation of feature importance. FIG. 4A) SHapley Additive explanations (SHAP) bee swarm plot indicating positive correlation of BS score and negative correlation of age with ploidy prediction. FIG. 4B) SHAP bee swarm plot indicating that TE score has the largest influence on the prediction of ploidy using logistic regression.

FIG. 5. Pearson correlation matrix of model input features. Morphological features, ICM, and TE scores are highly correlated; morphological features are not correlated highly with egg age or morphokinetic features.

FIG. 6. Receiver operator curves for STORK-A classification tasks and models. FIG. 6A) Receiver operator curves for STORK-A ANU vs. EUP. FIG. 6B) Receiver operator curves for STORK-A complex aneuploids (CA) vs. EUP+single aneuploids (SA). FIG. 6C) Receiver operator curves for STORK-A CA vs. EUP.

FIG. 7. Independent dataset validation. Trained STORK-A for aneuploid (ANU) vs. euploid (EUP) classification using images, maternal age, and morphokinetic parameters reported similar accuracies on the IVI Valencia test set and WCM-ES+ test set when compared to the STORK-A primary test set.

FIG. 8. Performance of CA vs EUP+SA with image, maternal age, morphokinetics, and BG on the primary test set and WCM Center of Reproduction and included images captured using the EmbryoScope+® (WCM-ES+).

FIG. 9. Performance of ANU vs EUP with image and maternal age on the primary test set, WCM-ES+, and IVI Valencia.

FIG. 10. Receiver operator curves for STORK-A classification tasks and models. FIG. 10A) ANU vs. EUP. FIG. 10B) CA vs. EUP+SA. FIG. 10C) CA vs. EUP (samples with complete data only*).

FIG. 11. Web interface. An exemplary automated platform that can be used in clinical settings to evaluate ploidy status as a support tool for embryologists, in accordance with various embodiments.

FIG. 12. Exemplary screenshots of the STORK-A application programming interface (API) with and without various input parameters expanded, in accordance with various embodiments. FIG. 12A) API with embryo image only. FIG. 12B) API with embryo image and the maternal age input field expanded. FIG. 12C) API with embryo image and the blastocyst score input field expanded. FIG. 12D) API with embryo image and the blastocyst grade input field expanded. FIG. 12E) API with embryo image and the blastocyst grade input field expanded (expansion). FIG. 12F) API with embryo image and the blastocyst grade input field expanded (inner cell mass). FIG. 12G) API with embryo image and the blastocyst grade input field expanded (trophectoderm). FIG. 12H) API with embryo image and the morphokinetic parameters input field expanded.

FIG. 13. Exemplary screenshots of the STORK-A API under different input parameters and with different outcomes, where the image on the left has no additional input parameters, while the image on the right has one or more additional input parameters, in accordance with various embodiments. FIG. 13A) Embryo ploidy prediction with embryo image alone. FIG. 13B) Embryo ploidy prediction with embryo image in combination with maternal age. FIG. 13C) Embryo ploidy prediction with embryo image in combination with morphological assessment (blastocyst score). FIG. 13D) Embryo ploidy prediction with embryo image in combination with morphological assessment (blastocyst grade). FIG. 13E) Embryo ploidy prediction with embryo image in combination with morphokinetic parameters. FIG. 13F) Embryo ploidy prediction with embryo image in combination with age and morphological assessment (blastocyst score). FIG. 13G) Embryo ploidy prediction with embryo image in combination with age and morphological assessment (blastocyst grade). FIG. 13H) Embryo ploidy prediction with embryo image in combination with age and morphokinetic parameters. FIG. 13I) Embryo ploidy prediction with embryo image in combination with age, blastocyst score, and morphokinetic parameters. FIG. 13J) Embryo ploidy prediction with embryo image in combination with age, blastocyst grade, and morphokinetic parameters.

### DETAILED DESCRIPTION

### I. Overview

This specification describes various exemplary embodiments of systems, software and methods for evaluating embryos by, for example, predicting embryo ploidy status. The disclosure, however, is not limited to these exemplary embodiments and applications or to the manner in which the exemplary embodiments and applications operate or are described herein.

As described herein, the present inventors have developed a non-invasive and automated method of embryo evaluation to predict blastocyst ploidy in a non-invasive manner as an improvement to traditional and inferior methods. This method uses artificial intelligence to non-invasively predict embryo ploidy status, and is referred to herein as “STORK-A”. Development of this method utilized a retrospective dataset of 10,378 embryos that consists of static images captured at 110 hpi, morphokinetic parameters, blastocyst morphological assessments, maternal age, and ploidy status. Independent and external datasets from WCM Center of Reproduction's EmbryoScope+® and IVI Valencia, Spain were used to test for generalizability. Several machine and deep learning models were developed to understand which features contribute to ploidy classification. Maternal age along with morphological assessment were strong predictors of embryo ploidy while morphokinetic parameters (tPnF-tSB) did not contribute to improving predictions.

STORK-A was found to predict aneuploid vs. euploid embryos with an accuracy of 69.3% (AUC=0.761) when, for example, using images, maternal age, morphokinetics, and blastocyst score. A second classification task trained to predict complex aneuploid vs. euploid and single aneuploid produced an accuracy of 74.0% (AUC=0.76 using an image, age, morphokinetic parameters, and blastocyst grade). A third classification task trained to predict complex aneuploid vs. euploid had an accuracy of 77.6% (AUC=0.847). STORK-A reported accuracies of 63.4% (AUC=0.702) and 65.7% (AUC=0.715) on the EmbryoScope+® and IVI Valencia datasets respectively when using an image, maternal age, and morphokinetic parameters, comparable to the STORK-A test set accuracy of 67.8% (AUC=0.737) showcasing generalizability.

As a proof-of-concept, STORK-A demonstrates a strong ability to correctly predict euploid and single aneuploid embryos in a non-invasive manner. This demonstrates the ability for STORK-A to be used alone or as a standardized supplementation to traditional (i.e. exclusively human, non-automated) methods of embryo selection and prioritization for implantation or recommendation for PGT-A. This study also shows the generalizability of STORK-A via the testing of independent datasets.

### II. Exemplary Descriptions of Terms

Unless otherwise defined, all terms of art, notations, and other scientific terms or terminology used herein are intended to have the meanings commonly understood by those of skill in the art to which this application pertains. Further, unless otherwise required by context, singular terms shall include pluralities and plural terms shall include the singular. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

It should be understood that any use of subheadings herein are for organizational purposes, and should not be read to limit the application of those subheaded features to the various embodiments herein. Each and every feature described herein is applicable and usable in all the various embodiments discussed herein and that all features described herein can be used in any contemplated combination, regardless of the specific example embodiments that are described herein. It should further be noted that exemplary description of specific features are used, largely for informational purposes, and not in any way to limit the design, subfeature, and functionality of the specifically described feature.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in various embodiments.

In addition, as the terms “on”, “attached to”, “connected to”, “coupled to”, or similar words are used herein, one element (e.g., a material, a layer, a substrate, etc.) can be “on”, “attached to”, “connected to”, or “coupled to” another element regardless of whether the one element is directly on, attached to, connected to, or coupled to the other element or there are one or more intervening elements between the one element and the other element. In addition, where reference is made to a list of elements (e.g., elements a, b, c), such reference is intended to include any one of the listed elements by itself, any combination of less than all of the listed elements, and/or a combination of all of the listed elements. Section divisions in the specification are for ease of review only and do not limit any combination of elements discussed.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, the use of these terms in the specification does not by itself connote any required priority, precedence, or order.

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within ten percent.

The term “ones” means more than one.

As used herein, the term “plurality” can be 2, 3, 4, 5, 6, 7, 8, 9, 10, or more.

As used herein, the term “set of” means one or more. For example, a set of items includes one or more items.

As used herein, the phrase “at least one of,” when used with a list of items, means different combinations of one or more of the listed items may be used and only one of the items in the list may be needed. The item may be a particular object, thing, step, operation, process, or category. In other words, “at least one of” means any combination of items or number of items may be used from the list, but not all of the items in the list may be required. For example, without limitation, “at least one of item A, item B, or item C” means item A; item A and item B; item B; item A, item B, and item C; item B and item C; or item A and C. In some cases, “at least one of item A, item B, or item C” means, but is not limited to, two of item A, one of item B, and ten of item C; four of item B and seven of item C; or some other suitable combination.

As used herein, the terms “comprise”, “comprises”, “comprising”, “contain”, “contains”, “containing”, “have”, “having”, “include”, “includes”, and “including” and their variants are not intended to be limiting, are inclusive or open-ended and do not exclude additional, unrecited additives, components, integers, elements or method steps. For example, a process, method, system, composition, kit, or apparatus that comprises a list of features is not necessarily limited only to those features but may include other features not expressly listed or inherent to such process, method, system, composition, kit, or apparatus. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

As used herein the specification, “a”, “an”, and “the,” may mean one or more. These terms generally refer to singular and plural references unless the context clearly dictates otherwise. As used herein in the claim(s), when used in conjunction with the word “comprising,” the words “a” or “an” may mean one or more than one. Some embodiments of the disclosure may consist of or consist essentially of one or more elements, method steps, and/or methods of the disclosure. It is contemplated that any method or composition described herein can be implemented with respect to any other method or composition described herein and that different embodiments may be combined. “A and/or B” is used herein to include all of the following alternatives: “A”, “B”, “A or B”, and “A and B”.

The use of the term “or” in the claims is used to mean “and/or” unless explicitly indicated to refer to alternatives only or the alternatives are mutually exclusive, although the disclosure supports a definition that refers to only alternatives and “and/or.” For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment. As used herein “another” may mean at least a second or more.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

As used herein, a “subject” or an “individual” includes animals, such as human (e.g., human individuals) and non-human animals. The term “non-human animals” includes all vertebrates, e.g., mammals, e.g., rodents, e.g., mice, non-human primates, and other mammals, such as e.g., rat, mouse, cat, dog, cow, pig, sheep, horse, goat, rabbit; and non-mammals, such as amphibians, reptiles, etc. A subject can be a mammal, preferably a human or humanized animal. The subject may be in need of prevention and/or treatment of a disease or disorder, such as infertility.

The term “patient,” as used herein, generally refers to a mammalian subject. The mammal can be a human, or an animal including, but not limited to an equine, porcine, canine, feline, ungulate, and primate animal. In one embodiment, the individual is a human. The methods and uses described herein are useful for both medical and veterinary uses. A “patient” is a human subject unless specified to the contrary.

“Treating” or treatment of a disease or condition refers to executing a protocol, which may include administering one or more drugs to an individual, such as a patient (or subject), in an effort to alleviate signs or symptoms of the disease. Desirable effects of treatment include decreasing the rate of disease progression, ameliorating or palliating the disease state, and remission or improved prognosis. Alleviation can occur prior to signs or symptoms of the disease or condition appearing, as well as after their appearance. Thus, “treating” or “treatment” may include “preventing” or “prevention” of disease or undesirable condition, such as infertility. In addition, “treating” or “treatment” does not require complete alleviation of signs or symptoms, does not require a cure, and specifically includes protocols that have only a marginal effect on the patient.

The term “therapeutically effective” as used throughout this application refers to anything that promotes or enhances the well-being of the subject with respect to the medical treatment of this condition. In some embodiments, administering a therapeutically effective amount results in treating the condition to some degree.

The term “sample,” as used herein, generally refers to a sample from a subject of interest and may include a biological sample of a subject. The sample may include a cell sample. The sample may include a cell line or cell culture sample. The sample can include one or more cells. The sample can include one or more microbes. The sample may include a nucleic acid sample or protein sample. The sample may also include a carbohydrate sample or a lipid sample. The sample may be derived from another sample. The sample may include a tissue sample, such as a biopsy, core biopsy, needle aspirate, or fine needle aspirate. The sample may include a fluid sample, such as a blood sample, urine sample, or saliva sample. The sample may include a skin sample. The sample may include a cheek swab. The sample may include a plasma or serum sample. The sample may include a cell-free or cell free sample. A cell-free sample may include extracellular polynucleotides. The sample may originate from blood, plasma, serum, urine, saliva, mucosal excretions, sputum, stool, or tears. The sample may originate from red blood cells or white blood cells. The sample may originate from feces, spinal fluid, CNS fluid, gastric fluid, amniotic fluid, cyst fluid, peritoneal fluid, marrow, bile, other body fluids, tissue obtained from a biopsy, skin, or hair.

Similarly, the terms “biological sample,” “biological specimen,” or “biospecimen” as used herein, generally refers to a specimen taken by sampling so as to be representative of the source of the specimen, typically, from a subject. A biological sample can be representative of an organism as a whole, specific tissue, cell type, or category or sub-category of interest. Biological samples may include, but are not limited to stool, synovial fluid, whole blood, blood serum, blood plasma, urine, sputum, tissue, saliva, tears, spinal fluid, tissue section(s) obtained by biopsy; cell(s) that are placed in or adapted to tissue culture; sweat, mucous, gastric fluid, abdominal fluid, amniotic fluid, cyst fluid, peritoneal fluid, pancreatic juice, breast milk, lung lavage, marrow, gastric acid, bile, semen, pus, aqueous humor, transudate, and the like including derivatives, portions and combinations of the foregoing. In some examples, biological samples include, but are not limited, to stool, biopsy, blood and/or plasma. In some examples, biological samples include, but are not limited, to urine or stool. Biological samples include, but are not limited, to biopsy. Biological samples include, but are not limited, to tissue dissections and tissue biopsies. Biological samples include, but are not limited, any derivative or fraction of the aforementioned biological samples. The biological sample can include a macromolecule. The biological sample can include a small molecule. The biological sample can include a virus. The biological sample can include a cell or derivative of a cell. The biological sample can include an organelle. The biological sample can include a cell nucleus. The biological sample can include a rare cell from a population of cells. The biological sample can include any type of cell, including without limitation prokaryotic cells, eukaryotic cells, bacterial, fungal, plant, mammalian, or other animal cell type, mycoplasmas, normal tissue cells, tumor cells, or any other cell type, whether derived from single cell or multicellular organisms. The biological sample can include a constituent of a cell. The biological sample can include nucleotides (e.g., ssDNA, dsDNA, RNA), organelles, amino acids, peptides, proteins, carbohydrates, glycoproteins, or any combination thereof. The biological sample can include a matrix (e.g., a gel or polymer matrix) comprising a cell or one or more constituents from a cell (e.g., cell bead), such as DNA, RNA, organelles, proteins, or any combination thereof, from the cell. The biological sample may be obtained from a tissue of a subject. The biological sample can include a hardened cell. Such hardened cells may or may not include a cell wall or cell membrane. The biological sample can include one or more constituents of a cell but may not include other constituents of the cell. An example of such constituents may include a nucleus or an organelle. The biological sample may include a live cell. The live cell can be capable of being cultured.

The term “marker” or “biomarker,” as used herein, generally refers to any measurable substance taken as a sample from a subject whose presence is indicative of some phenomenon. Non-limiting examples of such phenomenon can include a disease state, a condition, or exposure to a compound or environmental condition. In various embodiments described herein, markers or biomarkers may be used for diagnostic purposes (e.g., to diagnose a health state, a disease state). The term “biomarker” can be used interchangeably with the term “marker.”

The term “sequence,” as used herein, generally refers to a biological sequence including one-dimensional monomers that can be assembled to generate a polymer. Non-limiting examples of sequences include nucleotide sequences (e.g., ssDNA, dsDNA, and RNA), amino acid sequences (e.g., proteins, peptides, and polypeptides), and carbohydrates (e.g., compounds including Cm (H2O)n).

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number. If the degree of approximation is not otherwise clear from the context, “about” means either within plus or minus 10% of the provided value, or rounded to the nearest significant figure, in all cases inclusive of the provided value. In various embodiments, the term “about” indicates the designated value ±up to 10%, up to ±5%, or up to ±1%.

The term “training data,” as used herein generally refers to data that can be input into models, statistical models, algorithms and any system or process able to use existing data to make predictions.

As used herein, a “model” may include one or more algorithms, one or more mathematical techniques, one or more machine learning algorithms, or a combination thereof.

As used herein, “machine learning” may be the practice of using algorithms to parse data, learn from it, and then make a determination or prediction about something in the world. Machine learning uses algorithms that can learn from data without relying on rules-based programming. A machine learning algorithm may include a parametric model, a nonparametric model, a deep learning model, a neural network, a linear discriminant analysis model, a quadratic discriminant analysis model, a support vector machine, a random forest algorithm, a nearest neighbor algorithm, a combined discriminant analysis model, a k-means clustering algorithm, a supervised model, an unsupervised model, logistic regression model, a multivariable regression model, a penalized multivariable regression model, or another type of model.

As used herein, an “artificial neural network” or “neural network” (NN) may refer to mathematical algorithms or computational models that mimic an interconnected group of artificial nodes or neurons that processes information based on a connectionist approach to computation. Neural networks, which may also be referred to as neural nets, can employ one or more layers of nonlinear units to predict an output for a received input. Some neural networks include one or more hidden layers in addition to an output layer. The output of each hidden layer is used as input to the next layer in the network, i.e., the next hidden layer or the output layer. Each layer of the network generates an output from a received input in accordance with current values of a respective set of parameters. In the various embodiments, a reference to a “neural network” may be a reference to one or more neural networks.

A neural network may process information in two ways: when it is being trained it is in training mode and when it puts what it has learned into practice it is in inference (or prediction) mode. Neural networks learn through a feedback process (e.g., backpropagation) which allows the network to adjust the weight factors (modifying its behavior) of the individual nodes in the intermediate hidden layers so that the output matches the outputs of the training data. In other words, a neural network learns by being fed training data (learning examples) and eventually learns how to reach the correct output, even when it is presented with a new range or set of inputs. A neural network may include, for example, without limitation, at least one of a Feedforward Neural Network (FNN), a Recurrent Neural Network (RNN), a Modular Neural Network (MNN), a Convolutional Neural Network (CNN), a Residual Neural Network (ResNet), an Ordinary Differential Equations Neural Networks (neural-ODE), or another type of neural network.

It should be understood that while deep learning may be discussed in conjunction with various embodiments herein, the various embodiments herein are not limited to being associated only with deep learning tools. As such, machine learning and/or artificial intelligence tools generally may be applicable as well. Moreover, the terms deep learning, machine learning, and artificial intelligence may even be used interchangeably in generally describing the various embodiments of systems, software and methods herein.

In various embodiments, a deep learning, machine learning, and/or artificial intelligence system can take the form of one or more binary classification model. The binary classification model may include, for example, but is not limited to, a regression model. The binary classification model may include, for example, a penalized multivariable regression model that is trained to identify set of embryo features from a plurality of (or panel of) identified embryo feature options. The binary classification model may be trained to identify weight coefficients for embryo features, and those embryo features having non-zero weights or weight coefficients above a selected threshold (e.g., absolute weight coefficient above 0.0, 0.01, 0.05, 0.1, 0.015, 0.2, etc.) may be selected for inclusion in the set of embryo features.

### III. Overview of Exemplary Workflow

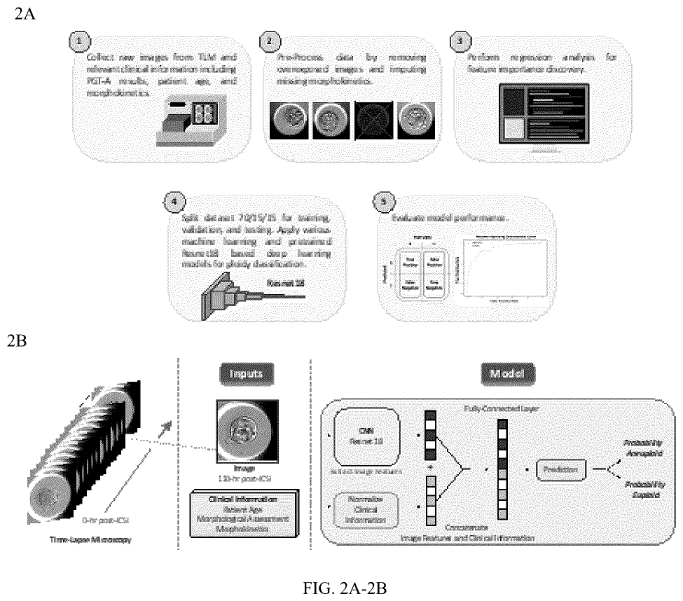

Exemplary workflows for various embodiments in accordance with the present invention, used for predicting ploidy status of an embryo, for example such as during in vitro fertilization, are shown in FIGS. 2A and 2B. For example, from time-lapse videos obtained for an embryo, a single static image at 110 hours post insemination (hpi) (focal plane 0) is analyzed, in combination with morphokinetic annotations, morphological assessments, maternal age, and/or associated PGT-A results for the embryo. Optionally, the dataset can be pre-processed to remove underexposed images by manual detection, and/or to input missing morphokinetic values using median imputation. LASSO and logistic regressions are then applied to clinical information to determine feature importance. Hyperparameters for the models can then be optimized through iterative training and once completed, the performance on the test set is evaluated. One or more deep learning model can be used for ploidy classification, where extracted image features can be concatenated with clinical information (maternal age, morphokinetic parameters, and one of three morphological assessments BG, BS, AIBS) before being passed on to a final fully-connected layer.

This example is described for illustrative purposes only, and other workflows are contemplated in accordance with various embodiments, involving additional steps and/or features, and/or removing certain steps and/or features used in illustrative exemplary embodiments.

The workflow may include various operations including, for example, sample collection, sample intake, sample preparation and processing, data analysis, and output generation.

Sample collection may include, for example, obtaining a biological sample of one or more subjects. The biological sample may take the form of a specimen obtained via one or more sampling methods. The biological sample may be a sample taken to obtain maternal, paternal, and/or embryonic genetic information. The biological sample may be obtained in any of a number of different ways. In various embodiments, the biological sample includes whole blood sample obtained via a blood draw. In various embodiments, the biological sample includes a cryopreserved whole blood sample or a cryopreserved sample. In other embodiments, the biological sample includes a set of aliquoted samples that includes, for example, a serum sample, a plasma sample, a blood cell (e.g., white blood cell (WBC), red blood cell (RBC)) sample, another type of sample, or a combination thereof. Biological samples may include nucleotides (e.g., ssDNA, dsDNA, RNA), organelles, amino acids, peptides, proteins, carbohydrates, glycoproteins, or any combination thereof.

Sample intake may include one or more various operations such as, for example, aliquoting, registering, processing, storing, thawing, and/or other types of operations.

Sample preparation and processing can include, for example pooling multiple samples into a single assay tube and “demultiplexing” after analysis (in silico) based on individual subject genotype—genotype-based demultiplexing of single cell analysis. Employing these types of approaches provides a rapidly scalable and economic workflow for research-phase single cell dataset building for multiple diseases. Sample preparation and processing can also include working with a single sample in a single assay tube.

Further, sample preparation and processing may include, for example, data acquisition based on a static image of an embryo. Sample preparation and processing may also include, for example, data acquisition based on clinical and/or morphological features for the embryo.

Data analysis may include, for example, machine and/or deep learning and regression analysis of a static image of an embryo and/or clinical and/or morphological features for the embryo. In some embodiments, data analysis also includes output generation. In other embodiments, output generation may be considered a separate operation from data analysis. Output generation may include, for example, generating final output based on the results of machine and/or deep learning and regression analysis of a static image of an embryo and/or clinical and/or morphological features for the embryo. In various embodiments, final output may be used for determining embryo ploidy. In various embodiments, final output may be used for determining the research, diagnosis, and/or treatment of infertility, by predicting embryo viability based on the embryo ploidy status, wherein an embryo having a stronger probability of being euploid has a higher probability of being viable. In various embodiments, final output may be used for determining the research, diagnosis, and/or treatment of infertility, by improving embryo selection during in vitro fertilization. In various embodiments, final output may be used for determining the research, diagnosis, and/or treatment of infertility, by selecting and/or prioritizing an embryo for preimplantation genetic testing for aneuploidy (PGT-A) biopsy and/or implantation during in vitro fertilization. In various embodiments, final output may be used for determining the research, diagnosis, and/or treatment of infertility, by combining the results of machine and/or deep learning and regression analysis of a static image of an embryo and/or clinical and/or morphological features for the embryo with traditional methods of embryo selection and prioritization for implantation and/or recommendation for PGT-A during in vitro fertilization. In various embodiments, final output may be used for determining the research, diagnosis, and/or treatment of infertility, by improving an outcome in a subject undergoing in vitro fertilization, comprising the method of any preceding claim, wherein an embryo predicted to be euploid is selected for embryo transfer during in vitro fertilization.

In various embodiments, final output is comprised of one or more outputs. Final output may take various forms. In some embodiments, the report can comprise a probability of embryo ploidy. For example, final output may be a report that includes, for example, a diagnosis output, a treatment output (e.g., a treatment design output, a treatment plan output, or combination thereof), analyzed data (e.g., relativized and normalized) or combination thereof. In some embodiments, final output may be sent to a remote system for processing. The remote system may include, for example, a computer system, a server, a processor, a cloud computing platform, cloud storage, a laptop, a tablet, a smartphone, some other type of mobile computing device, or a combination thereof.

In some embodiments, a final output may be sent to a remote system for processing in some examples. In other embodiments, a final output may be displayed on a graphical user interface in a display system for viewing by a human operator.

In other embodiments, any workflow as described herein may optionally exclude one or more of the operations described herein and/or may optionally include one or more other steps or operations other than those described herein (e.g., in addition to and/or instead of those described herein). Accordingly, any workflow as described herein may be implemented in any of a number of different ways to determine a probability of embryo ploidy and/or for use in the research, diagnosis, and/or treatment of, for example, infertility.

### IV. Predicting Embryo Quality Based on Probability of Embryo Ploidy

As women approach the end of their childbearing years, the incidence of aneuploid embryos, i.e. those that exhibit chromosomal abnormalities, increases. This often results in significant clinical consequences, such as infertility, miscarriage, and birth defects (1). As a result, there is an increasing trend for couples to conceive using assisted reproductive technologies. Experts in the field of reproductive medicine have geared their efforts towards selecting and transferring the single most viable embryo that will result in the live birth of one healthy child. Reductions in the number of embryos transferred confer several advantages for a patient, including decreased overall healthcare costs, minimizing potential complications, and reducing the mental, physical, and emotional tax of repeated implantation failures and pregnancy losses. This selection process presents itself as one of the chief challenges in the field of in vitro fertilization (IVF).

Both non-invasive and invasive methods for embryo selection are currently implemented in fertility clinics. Embryonic morphology assessment by an expert embryologist at discrete time points has been the predominant non-invasive means of evaluating embryo quality and subsequent selection for transfer (2,3). By focusing visual quality assessment on a set of morphological features correlated with viability, embryologists assign embryo quality through a rubric-like grading which emphasizes three aspects of blastocyst morphology: i) degree of blastocyst expansion and hatching status; ii) the inner cell mass (ICM); and iii) the trophectoderm (TE). More recently, Time-Lapse Microscopy (TLM) has gained traction as a supplemental tool for improved embryo selections. This technology allows embryologists to more readily monitor embryo development and carry out morphokinetic analysis which have been shown to be associated with improved implantation potential and pregnancy rate (4).

Morphological assessment and morphokinetic annotations are non-invasive. However, the two methods are time-consuming and suffer from intra-observer and inter-observer variability due to their inherent subjectivity (5-9).

Advancements in comprehensive chromosome screening technologies like preimplantation genetic testing for aneuploidy (PGT-A) provide a means of unbiased implantation potential by ensuring the transfer of a euploid, chromosomally normal embryo, which improves the chances of obtaining a successful live birth. This method of embryo selection is especially useful for patients of advanced maternal age who have an increased protentional for pregnancy failure. Although studies have demonstrated that PGT-A increases implantation potential and pregnancy rates by reducing the number of transferred aneuploid embryos, particularly amongst patients of advanced maternal age (10-12), evidence demonstrating the use of PGT-A amongst younger patients has not shown a large improvement from unbiopsied embryos.

While PGT-A can address issues of variability noted in morphological and morphokinetic analysis methods, several limitations remain. The invasive nature of PGT-A has given rise to moral and ethical issues and can result in a reduction in embryo quality and viability (13-16). In addition to these limitations, carrying out PGT-A is costly and time-consuming, requiring expertly trained embryologists to minimize the biopsy, reduce embryo cryogenic damage, and sophisticated molecular genetic diagnostic lab.

The assessment of embryo quality using non-invasive imaging techniques requires a high level of expertise and suffers from observer heterogeneity (14-16). Deep learning approaches have evolved in the past decade as a powerful tool for tasks such as image classification and have proven to be amenable to the analysis of embryonic imaging data. One particular and widely used deep learning approach for image classification tasks is the convolutional neural network (CNN). These networks are structured in different layers, and each layer consists of multiple image “filters”, which are used to extract important features either from the raw image pixels in the first layer, or an intermediate representation of the image in subsequent layers. The filters are optimized to perform the specific task of interest. Recent studies have leveraged the use of artificial intelligence to automate the selection of embryos for IVF.

Campbell et al. have explored the use of morphokinetics to determine if aneuploids displayed significant differences in temporal variables as euploids and subsequently model the risk of aneuploidy. They identified that tSB and tB were significantly different in the two classes (17). With these results, they created a simple recursive partitioning method to model the degree of aneuploid risk resulting in an AUC of 0.72. However, several studies that sought to identify statistical differences in morphokinetics between euploid and aneuploid embryos, similar to Campbell et al., resulted in conflicting outcomes with no single set of morphokinetic parameters consistently predicting embryo ploidy (18-24). This lack of consistency can be owed to inter-observer variability, the absence of standardized guidelines for annotating morphokinetic parameters, and culture protocols. More importantly, it places into question whether morphokinetics can reliably be used as alternatives to PGT-A for assessing ploidy status.

Chavez-Badiola et al. examined the ability of their deep learning model ERICA to rank embryos using PGT-A results and beta-HCG levels thereby establishing “good prognosis” and “poor prognosis” ground truth labels as well as maternal age (25). Ultimately, the group reported an accuracy of 70 (AUC=0.74), sensitivity of 0.54, and specificity of 0.86. On a per cycle basis, ERICA predicted a euploid embryo in the top rank in 15 out of 19 cases and a euploid in the top two in 18 out of 19 cases. Within the study, the group does not differentiate between single and complex aneuploids, assumes that beta-HCG levels of 20 mUI/ml or more on the 7th day of embryo transfer is euploid, and the dataset size is small for developing a robust and generalizable model.

A more recent study by Lee et al. deviated from the common approach of using a single static image and instead utilized full-length time-lapse videos as proof of concept for ploidy prediction (26). Using a two-stream I3D architecture with videos that span day 1 to day 5 of development, the group achieved an AUC of 0.74 when predicting aneuploid vs. euploid and mosaic embryos. The authors note several limitations of their studying including dataset size (n=690), a lack of inclusion of embryos from patients older than 37, and an unbalanced dataset. One additional limitation in terms of applicability and deployment in the clinic is the use of time-lapse microscopy. While there are certainly advantages to using time-lapse machinery, its use across clinics is limited, and this would subsequently limit the use of this method.

The invention described herein relates to a deep learning method called STORK-A, which utilizes images captured by time-lapse microscopy and clinical information including maternal age, morphokinetic parameters, and morphological assessment, to accurately predict human embryo ploidy. The purpose of STORK-A is to aid clinicians in the selection and prioritization of embryos for PGT-A biopsy or implantation in a cost-efficient, standardized, and non-invasive manner.

Analysis and model development included the use of 10,378 embryos, all with PGT-A results, from 1,385 patients. Several clinical features were used to develop predictions, including maternal age ranging from 21 to 48 (mean=36.98, s.d.=4.62), morphokinetic parameters, morphological assessment, and images captured at 110 hours post insemination (hpi).

In this study, it was observed that among the most important features for ploidy classification was maternal age at the time of oocyte retrieval, which is known to correlate with the incidence of aneuploidy. Additionally, morphological features showcased a significant role in ploidy prediction as embryologist-derived improved model performance. Conversely, morphokinetics were found to play a less significant role in classification. The median imputation of missing morphokinetic parameters did not alter these conclusions, which we verified by retraining all machine and deep learning models (Tables 10 and 11).

Several recent studies have attempted to mimic the skill and experience of trained embryologists in assessing embryo quality while simultaneously improving consistency and reducing bias through the development of unbiased and automated embryo selection tools using deep learning. Khosravi et al. developed STORK, an embryo morphological assessment model based on the Veeck and Zaninovic Scale, used transfer learning with Inception-v1.9 STORK predicted embryo quality with near-perfect accuracy and demonstrated that its good-quality predictions were associated with better live birth outcomes. Chen et al. published a similar study in which they aimed to automate embryo grading using deep learning on a ResNet50 architecture (33). Bormann et al. developed a model for rank-based selection of embryos based on quality in addition to assessing the implantation potential of embryos (34). While automated morphological assessment is useful for developing a standardized method of grading embryo quality, these methods do not address the need to non-invasively predict the ploidy status of embryos as a means of prioritizing and selecting embryos with the highest implantation potential.

Our deep learning approach, STORK-A, demonstrated an ability to classify the ploidy status of embryos in three distinct classification tasks: ANU vs. EUP, CA vs. EUP+SA, and CA vs. EUP. The best models of these three classifiers incorporated an image, maternal age (age at the time of oocyte retrieval), morphokinetic parameters, and morphological assessment (BG or BS). Overall, the use of static images of embryos at 110 hpi to predict ploidy status did not markedly improve the performance of STORK-A when comparing machine and deep learning models across three classification tasks. This can be due to the images capturing embryos in different stages of development at 110 hpi in which case the deep learning models are learning to distinguish features that differ between morulas and blastocysts, rather than differences only between blastocysts as is the case for morphological assessment.

We also show that a standardized deep learning regression model to predict AIBS offers an improvement over age alone in the machine and deep learning models. While the performance gain is not quite as high compared to using embryologist-derived morphological assessment, STORK-A has the advantages of being non-invasive as well as automated. This work demonstrates as a proof-of-concept that artificial intelligence can approach the performance of expert annotators in providing useful information about embryo implantation potential. The inclusion of temporal and spatial information from videos of embryo development, rather than a single static image, have been found to yield improved ploidy predictive performance.

Several limitations arose in the study that warrant mentioning. First, embryos in the dataset used to train, validate, and test STORK-A were previously selected by embryologists as candidates for PGT-A based on their morphology. Those that were not biopsied for PGT-A were therefore not included. In other studies, unused embryos have been included and labeled as negative results. This work deviated from other studies to gain more confidence in STORK-A's ability to detect ploidy, however, this does have the potential to bias the dataset. An ideal dataset would include PGT-A results for all embryos regardless of their morphological quality.

Another limitation was the use of images captured only by time-lapse microscopy limiting generalizability. Time-lapse machines are costly, and few clinics utilize this technology. However, because STORK-A makes use of single static images, future development will incorporate images of embryos captured using different imaging modalities from different clinics, which will improve generalizability.

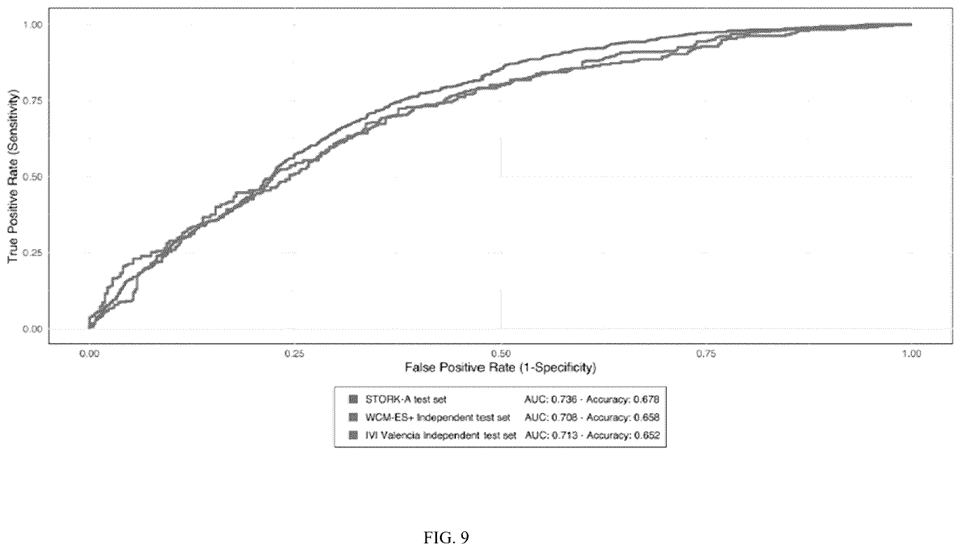

Morphokinetic annotations, which require time-lapse machinery, and morphological assessments were incorporated into STORK-A, thereby introducing human bias which could limit generalizability. An ideal artificial intelligence model would not be trained using unstandardized and subjective observations like morphokinetic parameters and morphological assessment, instead, it would be trained on standardized and reproducible data. To this end, we attempted to utilize an AI-driven predicted blastocyst score, AIBS, which is standardized and reproducible, to address this limitation. It should be noted, however, that the accuracies of STORK-A classifiers using an image and maternal age only show decreases of 2-4% compared to those incorporating subjective morphokinetic parameters and morphological assessments and were generalizable to the independent test sets (FIG. 9).

Lastly, Next Generation Sequencing can distinguish genetic sequences of euploids, several types of aneuploids, and high- or low-level mosaic embryos. However, differences in mosaic reporting across genetic labs introduce limitations to generalizability when these results are used as ground truth labels. In this study, PGT-A results for the primary dataset from WCM and the WCM-ES+ independent dataset were generated from the same genetics lab. Between both datasets, 719 embryos had detailed genetic information, and of those, only 32 were mosaic and categorized as euploid. The IVI Valencia, Spain independent dataset includes PGT-A results from a different genetics lab that did not provide detailed sequencing information of embryos and instead determined embryos to be euploid or not euploid. Due to the narrow reporting of sequencing information, mosaicism was not taken into consideration during model development and therefore cannot be assessed by STORK-A. Therefore, the binary classification scheme of STORK-A introduces a limitation as mosaic embryos with high implantation potential could be misclassified.

This study demonstrates a future role for STORK-A in the fertility clinic. As described herein, STORK-A can be used as a decision-making tool that provides a standardized, non-invasive, and cost-efficient means of selecting and prioritizing high-quality embryos for PGT-A biopsy or transfer to patients as opposed to the traditional methods like morphological assessment, which are biased and subjective. STORK-A for CA vs. EUP+SA in particular would be very beneficial in the clinic. The high specificity of 80.1% can assist in identifying euploid and SA embryos without misclassifying a large number of CA when prioritizing embryos for biopsy or transfer. An embryologist could confidently assess an embryo as truly being euploid or single aneuploid with a negative predictive value of 82.3%.

The question about the actual benefit of PGT-A is pertinent to this study. Although PGT-A can detect chromosomal abnormalities with great accuracy, a recent Cochrane Review has found that there is insufficient evidence to support its use as it has not led to increased pregnancy rates or live birth outcomes (35). If the ultimate goal of developing assistive reproductive technologies for IVF is to reduce a patient's time to pregnancy and improved live birth outcomes, that should be the gold standard. However, as it stands embryo selection is still critical to this outcome, and embryos with the greatest implantation potential must be selected or prioritized while those with low implantation potential are deprioritized. Current widespread screening methods like morphological assessments for embryo selection are unstandardized, and subjective except for PGT-A. It is this type of standardization, free of variability, that is necessary for the development of methods to prioritize and select embryos that are consistent across clinics. For this reason, we elected to use PGT-A results as the ground truth labels for the development of our models. STORK-A is poised to provide standardized embryo selection and prioritization in a manner that is non-invasive, cost-efficient, and time-efficient.

### V. Neural Networks

Image classification/recognition generally requires accepting an input image and outputting a class or a probability of classes that best describes the image. This can be done using a computer system equipped with a processing engine, which utilizes algorithms, to process the input image and outputting a result. Image detection can also utilize a similar processing engine, whereby the system accepts an input image and identifies objects of interest within that image with a high level of accuracy using the algorithms pre-programmed into the processing engine.

Regarding the input image, the system will generally orient the input image as an array of pixel values. These pixel values, depending on the image resolution and size, will be an array of numbers corresponding to (length)×(width)×(# of channels). The number of channels can also be referred to as the depth. For example, the array could be L×W×Red Green Blue color model (RBG values). The RGB would be considered three channels, each channel representing one of the three colors in the RGB color model. For example, the system can generally characterize a 20×20 image with a representative array of 20×20×3 (for RGB), with each point in the array assigned a value (e.g., 0 to 255) representing pixel intensity. Given this array of values, the processing engine can process these values, using its algorithms, to output numbers that describe the probability of the image being a certain class (e.g., 0.80 for cell, 0.15 for cell wall, and 0.05 for no cell).

A deep neural network (DNN) generally, such as a convolutional neural network (CNN), generally accomplishes an advanced form of image processing and classification/detection by first looking for low level features such as, for example, edges and curves, and then advancing to more abstract (e.g., unique to the type of images being classified) concepts through a series of convolutional layers. A DNN/CNN can do this by passing an image through a series of convolutional, nonlinear, pooling (or downsampling, as will be discussed in more detail below), and fully connected layers, and get an output. Again, the output can be a single class or a probability of classes that best describes the image or detects objects on the image.

Regarding layers in a CNN, for example, the first layer is generally a convolutional layer (Conv). This first layer will process the image's representative array using a series of parameters. Rather than processing the image as a whole, a CNN will analyze a collection of image sub-sets using a filter (or neuron or kernel). The sub-sets will include a focal point in the array as well surrounding points. For example, a filter can examine a series of 5×5 areas (or regions) in a 32×32 image. These regions can be referred to as receptive fields. Since the filter must possess the same depth of the input, an image with dimensions of 32×32×3 would have a filter of the same depth (e.g., 5×5×3). The actual step of convolving, using the exemplary dimensions above, would involve sliding the filter along the input image, multiplying filter values with the original pixel values of the image to compute element wise multiplications, and summing these values to arrive at a single number for that examined portion of the image.

After completion of this convolving step, using a 5×5×3 filter, an activation map (or filter map) having dimensions of 28×28×1 will result. For each additional layer used, spatial dimensions are better preserved such that using two filters will result in an activation map of 28×28×2. Each filter will generally have a unique feature it represents (e.g., colors, edges, curves, etc.) that, together, represent the feature identifiers required for the final image output. These filters, when used in combination, allow the CNN to process an image input to detect those features present at each pixel. Therefore, if a filter serves as a curve detector, the convolving of the filter along the image input will produce an array of numbers in the activation map that correspond to high likelihood of a curve (high summed element wise multiplications), low likelihood of a curve (low summed element wise multiplications) or a zero value where the input volume at certain points provided nothing that would activate the curve detector filter. As such, the greater number of filters (also referred to as channels) in the Conv, the more depth (or data) that is provided on the activation map, and therefore more information about the input that will lead to a more accurate output.

Balanced with accuracy of the CNN is the processing time and power needed to produce a result. In other words, the more filters (or channels) used, the more time and processing power needed to execute the Conv. Therefore, the choice and number of filters (or channels) to meet the needs of the CNN method are specifically chosen to produce as accurate an output as possible while considering the time and power available.

To enable further a CNN to detect more complex features, additional Conv layers can be added to analyze what outputs from the previous Conv layer (i.e., activation maps). For example, if a first Conv layers looks for a basic feature such as a curve or an edge, a second Conv layer can look for a more complex feature such as shapes, which can be a combination of individual features detected in an earlier Conv layer. By providing a series of Conv layers, the CNN can detect increasingly higher-level features to arrive eventually at the specific desired object detection. Moreover, as the Conv layers stack on top of each other, analyzing the previous activation map output, each Conv layer in the stack is naturally going to analyze a larger and larger receptive field by virtue of the scaling down that occurs at each Conv level, thereby allowing the CNN to respond to a growing region of pixel space in detecting the object of interest.

A CNN architecture generally consists of a group of processing blocks, including at least one processing block for convoluting an input volume (image) and at least one for deconvolution block (or transpose convolution). Additionally, the processing blocks can include at least one pooling block and unpooling block. Pooling blocks can be used to scale down an image in resolution to produce an output available for Conv. This can provide computational efficiency (efficient time and power), which can in turn improve actual performance of the CNN. Those these pooling, or subsampling, blocks keep filters small and computational requirements reasonable, these blocks coarsen the output (can result in lost spatial information within a receptive field), reducing it from the size of the input by a factor equal to the pixel stride of the receptive fields of the output units.

Unpooling blocks can be used to reconstruct a these coarse outputs to produce an output volume with the same dimensions as the input volume. An unpooling block can be considered a reverse operation of a convoluting block to return an activation output to the original input volume dimension.

However, the unpooling process generally just simply enlarges the coarse outputs into a sparse activation map. To avoid this result, the deconvolution block densifies this sparse activation map to produce both and enlarged and dense activation map that eventually, after any further necessary processing, a final output volume with size and density much closer to the input volume. As a reverse operation of the convolution block, rather than reducing multiple array points in the receptive field to a single number, the deconvolution block associate a single activation output point with a multiple outputs to enlarge and densify the resulting activation output.

It should be noted that while pooling blocks can be used to scale down an image and unpooling blocks can be used to enlarge these scaled down activation maps, convolution and deconvolution blocks can be structured to both convolve/deconvolve and scale down/enlarge without the need for separate pooling and unpooling blocks.

The pooling and unpooling process can be limited depending on the objects of interest being detected in an image input. Since pooling generally scales down an image by looking at sub-image windows without overlap of windows, there is a clear loss in spatial info as the scaling down occurs.

A processing block can include other layers that are packaged with a convolutional or deconvolutional layer. These can include, for example, a rectified linear unit layer (ReLU) or exponential linear unit layer (ELU), which are activation functions that examine the output from a Conv layer in its processing block. The ReLU or ELU layer acts as a gating function to advance only those values corresponding to positive detection of the feature of interest unique to the Conv layer its processing block.

Given a basic architecture, the CNN is then prepared for a training process to hone its accuracy in image classification/detection (of objects of interest). Using training data sets, or sample images used to train the CNN so that it updates its parameters in reaching an optimal, or threshold, accuracy, a process called backpropagation (backprop) occurs. Backpropagation involves a series of repeated steps (training iterations) that, depending on the parameters of the backprop, either will slowly or quickly train the CNN. Backprop steps generally include forward pass, loss function, backward pass, and parameter (weight) update according to a given learning rate. The forward pass involves passing a training image through the CNN. The loss function is a measure of error in the output. The backward pass determines the contributing factors to the loss function. The weight update involves updating the parameters of the filters to move the CNN towards optimal. The learning rate determines the extent of weight update per iteration to arrive at optimal. If the learning rate is too low, the training may take too long and involve too much processing capacity. If the learning rate is too fast, each weight update may be too large to allow for precise achievement of a given optimum or threshold.

The backprop process can cause complications in training, thus leading to the need for lower learning rates and more specific and carefully determined initial parameters upon start of training. One such complication is that, as weight updates occur at the conclusion of each iteration, the changes to the parameters of the Conv layers amplify the deeper the network goes. For example, if a CNN has a plurality of Conv layers that, as discussed above, allows for higher-level feature analysis, the parameter update to the first Conv layer is multiplied at each subsequent Conv layer. The net effect is that the smallest changes to parameters have large impact depending on the depth of a given CNN. This phenomenon is referred to as internal covariate shift.

It should be noted that even though CNNs are spoken about in detail above, the various embodiments discussed herein could utilize any neural network type or architecture.

### VI. Computer-Implemented System

In various embodiments, the systems and methods for determining embryo ploidy status can be implemented via computer software or hardware.

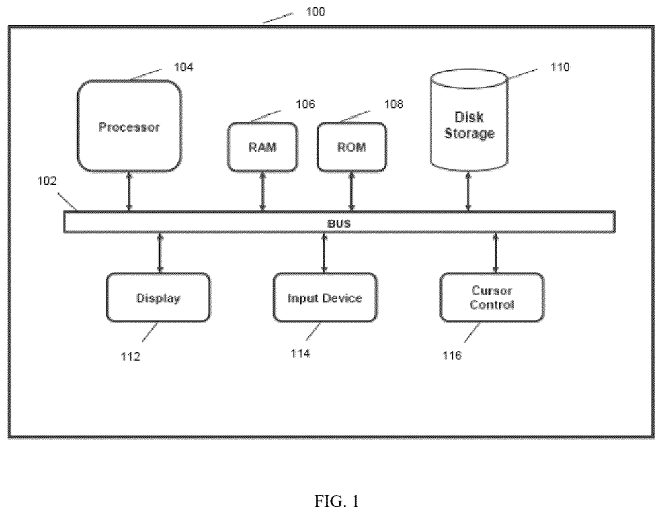

FIG. 1 is a block diagram illustrating a computer system 100 upon which embodiments of the present teachings may be implemented. In various embodiments of the present teachings, computer system 100 can include a bus 102 or other communication mechanism for communicating information and a processor 104 coupled with bus 102 for processing information. In various embodiments, computer system 100 can also include a memory, which can be a random-access memory (RAM) 106 or other dynamic storage device, coupled to bus 102 for determining instructions to be executed by processor 104. Memory can also be used for storing temporary variables or other intermediate information during execution of instructions to be executed by processor 104. In various embodiments, computer system 100 can further include a read only memory (ROM) 108 or other static storage device coupled to bus 102 for storing static information and instructions for processor 104. A storage device 110, such as a magnetic disk or optical disk, can be provided and coupled to bus 102 for storing information and instructions.

In various embodiments, computer system 100 can be coupled via bus 102 to a display 112, such as a cathode ray tube (CRT) or liquid crystal display (LCD), for displaying information to a computer user. An input device 114, including alphanumeric and other keys, can be coupled to bus 102 for communication of information and command selections to processor 104. Another type of user input device is a cursor control 116, such as a mouse, a trackball or cursor direction keys for communicating direction information and command selections to processor 104 and for controlling cursor movement on display 112. This input device 114 typically has two degrees of freedom in two axes, a first axis (i.e., x) and a second axis (i.e., y), that allows the device to specify positions in a plane. However, it should be understood that input devices 114 allowing for 3-dimensional (x, y and z) cursor movement are also contemplated herein.

Consistent with certain implementations of the present teachings, results can be provided by computer system 100 in response to processor 104 executing one or more sequences of one or more instructions contained in memory 106. Such instructions can be read into memory 106 from another computer-readable medium or computer-readable storage medium, such as storage device 110. Execution of the sequences of instructions contained in memory 106 can cause processor 104 to perform the processes described herein. Alternatively, hard-wired circuitry can be used in place of or in combination with software instructions to implement the present teachings. Thus, implementations of the present teachings are not limited to any specific combination of hardware circuitry and software.

The term “computer-readable medium” (e.g., data store, data storage, etc.) or “computer-readable storage medium” as used herein refers to any media that participates in providing instructions to processor 104 for execution. Such a medium can take many forms, including but not limited to, non-volatile media, volatile media, and transmission media. Examples of non-volatile media can include, but are not limited to, dynamic memory, such as memory 106. Examples of transmission media can include, but are not limited to, coaxial cables, copper wire, and fiber optics, including the wires that comprise bus 102.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, another memory chip or cartridge, or any other tangible medium from which a computer can read.

In addition to computer-readable medium, instructions or data can be provided as signals on transmission media included in a communications apparatus or system to provide sequences of one or more instructions to processor 104 of computer system 100 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, etc.

It should be appreciated that the methodologies described herein, flow charts, diagrams and accompanying disclosure can be implemented using computer system 100 as a standalone device or on a distributed network or shared computer processing resources such as a cloud computing network.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

In various embodiments, the methods of the present teachings may be implemented as firmware and/or a software program and applications written in conventional programming languages such as C, C++, Python, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as computer system 100, whereby processor 104 would execute the analyses and determinations provided by these engines, subject to instructions provided by any one of, or a combination of, memory components 106/108/110 and user input provided via input device 114.

Although specific embodiments and applications of the disclosure have been described in this specification, these embodiments and applications are exemplary only, and many variations are possible. Having described the invention in detail, it will be apparent that modifications, variations, and equivalent embodiments are possible without departing from the scope of the invention defined in the appended claims. Furthermore, it should be appreciated that all examples in the present disclosure are provided as non-limiting examples.

### VII. EXAMPLES

The following non-limiting examples are provided to further illustrate embodiments of the invention disclosed herein. It should be appreciated by those of skill in the art that the techniques disclosed in the examples that follow represent approaches that have been found to function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

### Example 1

### Methods

The methods used in Examples 2-9 are described below.

### Source Data

In this retrospective study, machine and deep learning approaches were tested to develop a novel model for the prediction of ploidy status. The de-identified dataset consisted of static time-lapse images (500×500 pixels) at 110 hours post-insemination (hpi), maternal age at the time of oocyte retrieval, blastocyst grade (BG), blastocyst score (BS), morphokinetic parameters ranging from pro-nuclear fading (tPnF) to the time of the start of blastulation (tSB), and PGT-A results. The dataset encompassed 10,378 human blastocysts (Day 5 n=3,994; Day 6 n=6,384) collected from 1,385 patients from 2012 to 2017 at the Center of Reproductive Medicine at Weill Cornell Medicine.

Images and morphokinetics were captured using an Embryoscope® time-lapse imaging instrument. Images at 110 hpi were utilized because this was the average time that embryologists assessed the morphological quality of embryos and biopsied cells for PGT-A. At 110 hours, the developmental stage of embryos varies between the morula and blastocyst stage; therefore, this time point accounts for temporal differences in blastocyst formation. Four expertly trained embryologists at the Center of Reproductive Medicine at Weill Cornell Medicine manually annotated morphokinetic parameters and assigned BGs using the Veeck and Zaninovic grading system, which includes assessments of the ICM, TE, and Expansion (27). BSs were derived from a system that converts TE, ICM, and Expansion grades, into numerical values established by Zhan et al. (28). BS also takes into consideration the day of blastocyst formation, i.e. Day 5 vs. Day 6, when calculating the score.

Embryos were biopsied for PGT-A on Day 5 if the morphological grade was a 2BB or better. The remaining embryos were biopsied on Day 6 so long as they had reached the blastocyst stage. In instances where patients had a limited number of viable embryos, embryos were biopsied on Day 6 if they were in the morula stage or cavitating morula stage. PGT-A results were categorized into two classes aneuploids (ANU) (n=5,953) and euploids (EUP) (n=4,425) and were used as ground-truth labels for the ploidy prediction task. The aneuploid class could be further stratified into single aneuploids (SA) (n=2,944) and complex aneuploids (CA) (n=3,009). SA exhibit one chromosomal abnormality, whereas CA exhibits two or more chromosomal abnormalities. Livebirth outcomes and fetal-heart results for 1,638 transferred embryos were also included. Of the 10,378 embryos in the dataset, 2,426 (SA n=697; CA n=951; EUP n=778) had at least one or more morphokinetic parameters missing. For these instances of missing data, median imputation was utilized to replace missing morphokinetic parameters with median values. Additionally, 12 static images that were underexposed were removed from the dataset.

To confirm the generalizability of STORK-A, independent datasets from Weill Cornell Medicine and IVI Valencia were sourced. The Weill Cornell Medicine dataset, captured from 2018-2019 using the Embryoscope+® (WCM-ES+), consisted of 841 embryos including single aneuploids (n=170), complex aneuploids (n=261), and euploids (n=410), maternal age, morphokinetic parameters, and morphological assessment. The IVI Valencia dataset from 2018 consisted of 554 embryos, including aneuploids (n=319) and euploids (n=235), maternal age, and morphokinetic parameters. Morphokinetic parameters were manually annotated by embryologists at IVI Valencia. The morphological criteria to select embryos for biopsy resembles that used at Weill Cornell Medicine.

### Clinical Feature Importance Determination Using LASSO Regression

LASSO regression, with a regularization term, C, of weight 0.01, was performed using the scikit-learn package in python. The regularization term was determined using a cross-validation grid search on each split of the training set with average precision as the scoring metric with regularization terms starting at 0.0001 and increasing by a factor of 10 to a regularization term of 10. Maternal age, morphological assessment, and morphokinetic parameters were used for the analysis. 5-fold cross-validation was performed, and 95% confidence intervals (CI) were calculated for each of the coefficients associated with each feature. All features were Z-score normalized based on the training set for each of the cross-validation splits.

### Morphological Feature Importance Using Logistic Regression

Logistic regression was performed using the scikit-learn package with the following parameters, penalty=‘12’, C=10, and class_weight=‘balanced’, where the penalty is the type of regularization, C is the strength of regularization, and class_weight is the method for weighting the loss function to account for class imbalance. Two different sets of features were analyzed based on previous LASSO-regression results, maternal age, and blastocyst score, as well as maternal age and each blastocyst grade component (TE, ICM, Expansion). All grades were converted from letter to numerical grades, such that A=6, A-=5, etc. Intermediate scores, i.e., 1-2 for expansion grades, were given an intermediate score, in this case, 1.5. Z-score normalization and median imputation were performed identically as what was performed for LASSO regression. Three subsets of data based on ploidy type were compared, 1) ANU vs. EUP, 2) CA vs. SA+EUP, and 3) CA vs. EUP. Five-fold cross-validation was performed, and validation set accuracy is reported (mean and 95% CI bounds). In addition, univariate analysis for each component of the blastocyst grades was performed. The weights for each feature were recorded to analyze feature importance from the logistic regression models and SHapley Additive explanations (SHAP) values were also used to validate feature importance (29).

### Blastocyst Score Prediction

The embryologist-derived morphological assessments used in the study are subject to variability. To standardize morphological assessment, a BS regression model was trained using a deep learning model based on the ResNet18 architecture pre-trained on ImageNet and performed using Pytorch. The ResNet18 architecture was modified to perform a regression task by adding two fully-connected layers, both of which were fine-tuned to perform BS regression. Using the primary dataset, the model was trained and validated on images of embryos at 110 hpi and used embryologist-derived blastocyst scores as ground truth labels to produce AIBS, artificial intelligence blastocyst scores. The model was trained for 20 epochs, with a batch size of 32, Adam optimization with a learning rate of 0.001, and MSE Loss. To ensure the model did not overfit on the training data, early-stopping, with patience=2, was implemented.

### Machine Learning and Deep Learning

Extreme Gradient Boost Decision Tree (XGBoost), k-nearest neighbor (k-NN), support vector machine (SVM), and Random Forest were trained using 5-fold cross-validation and tested in R using the caret package. Clinical features were used for input with ploidy status determined by PGT-A as the predicted outcome.

To exploit the spatial features of static embryo images for ploidy classification, STORK-A was trained, validated, and tested using Pytorch. STORK-A is based on a ResNet18 CNN architecture pre-trained on ImageNet30. The ResNet18 architecture was modified to concatenate features from images with clinical features before being passed on to two fully-connected layers that were fine-tuned to output the predicted probabilities of a binary classification task (FIG. 2). Several models were created to assess combinations of feature input to identify which features performed best. Models were trained for 20 epochs, with a batch size of 32, Adam optimization with a learning rate of 0.0001, and Cross Entropy Loss. Image augmentation was used to increase the magnitude of the training set and included a random resized crop of size 224×224 pixels and random horizontal and vertical flips.

A random 70/15/15 training, validation, and test (primary) split was applied and consistent across all models by using a set seed for reproducibility and comparison across all models. Clinical features in the training, validation and test sets of both machine and deep learning models were z-scored normalized to the training set. Several subsets of the data were identified to address different classification tasks including 1) ANU (SA+CA) vs. EUP, 2) CA vs. SA+EUP, and 3) CA vs. EUP. To address issues of class imbalance in the training set, the minority class was oversampled.

All binary classification thresholds for both machine learning and deep learning models were maintained at 50%. For example, in the ANU vs. EUP classification task a sample was classified as aneuploid when the probability was greater than 50% and euploid when the probability was less than 50%.

### Statistical Analysis

The predictive performance of the machine learning models on the primary test sets was assessed using the accuracy, 95% confidence interval, and positive predictive value (PPV) for each model. The performance of the STORK-A deep learning models on the primary test set was measured using accuracy, 95% confidence interval, PPV, negative predictive value (NPV), receiver operator curves (ROC), and AUC. Sensitivity and specificity for STORK-A were reported for the models with the best performance for each classification task. The independent and external datasets, WCM-ES+ and IVI Valencia, used to assess the generalizability of STORK-A were compared measuring accuracy, AUC, PPV, and NPV.

To gain further insight into the specific demographic performance of STORK-A with the task of classifying ANU vs. EUP, the primary test set was stratified across maternal ages and the day of blastocyst formation for post hoc analysis. Predictions from the primary test set were separated into Day 5 and Day 6 embryos, and four age groups, age <35, 35<Age <37, 37<Age <39, and Age >39, based on a similar procedure used by Irani et al. (31). The accuracy for each of these demographics within the primary test were then reported. Understanding the role of age associated with aneuploidy, embryos from patients aged 37 to 42 in the primary test set was separated into individual groups to identify the optimal classification thresholds that maximize the sum of sensitivity and specificity. Lastly, a post hoc analysis of fetal heart and live birth rates was conducted to explore the association between STORK-A and positive outcomes in the primary test set and stratified by age group.

### Example 2

### LASSO Regression for Clinical Feature Importance

LASSO regression, or logistic regression with L1 regularization, was used to introduce sparsity into the model prediction and improve the interpretability of clinical features and their contributions to ploidy prediction. 5-fold cross-validation was performed for three different tasks, 1) ANU vs. EUP, 2) CA vs. EUP, and 3) CA vs. SA+EUP. Features include maternal age, morphokinetics from pro-nuclear fading (tPnF) to the start of blastulation (tSB), blastocyst grade (BG) which includes assessment of TE, ICM, and the degree of expansion, and blastocyst score (BS). The data sets used for model development are shown in Table 1.

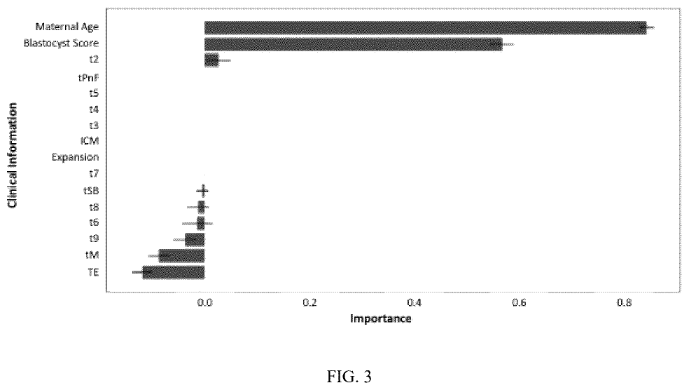

We found that, of these features, maternal age and blastocyst score rank among the highest contributors when predicting ploidy status prediction for all three tasks (FIG. 3). This is closely followed by trophectoderm grading from the blastocyst grade. We also observe the morphokinetics skew both positively and negatively in terms of importance and tend to have the least influence on ploidy prediction in this model. This result follows previously published work that shows age and blastocyst score correlated with ploidy (28).

TABLE 1
Datasets for model development: The primary datasets were used
for training, validating, and testing the models. Model development
focused on three individual classification tasks 1) ANU vs.
EUP, 2) CA vs. SA + EUP, and 3) CA vs. EUP. The primary
training set for each classification task includes oversampling
of the minority class to address the class imbalance. Two
independent datasets, WCM-ES+ and IVI Valencia were used
for testing the generalizability of the models.

| Classification Task | Embryo Ploidy Distribution | | | |
|---|---|---|---|---|
| 1. ANU vs. EUP | ANU (SA + CA) | CA | SA | EUP |
| Primary Training | 4,168 | 2,071 | 2,097 | 4,168 |
| Primary Validation | 893 | 467 | 426 | 664 |
| Primary Test | 892 | 471 | 421 | 663 |
| WCM-ES+ | 431 | 261 | 170 | 410 |
| IVI Valencia | 319 | . . . | . . . | 235 |
| 2. CA vs. EUP + SA | CA | EUP + SA | EUP | SA |
| Primary Training | 5,159 | 5,159 | 3,098 | 2,061 |
| Primary Validation | 451 | 1106 | 664 | 442 |
| Primary Test | 451 | 1104 | 663 | 441 |
| WCM-ES+ | 261 | 580 | 410 | 170 |
| 3. CA vs. EUP | CA | EUP | | |
| Primary Training | 3,098 | 3,098 | | |
| Primary Validation | 451 | 664 | | |
| Primary Test | 451 | 663 | | |
| WCM-ES+ | 261 | 410 | | |

### Example 3

### Logistic Regression for Morphological Component Analysis

Additional logistic regression models were performed to provide a more granular assessment of blastocyst score influence on ploidy prediction due to its feature importance from LASSO regression. When comparing the ploidy prediction performance of a logistic regression model with L2 regularization using maternal age and BS, compared to maternal age and the three components of blastocyst grade, TE, ICM, and Expansion, accuracies were similar (Table 2). In addition, results from this model were comparable to results obtained using the entire clinical feature space for CA vs. SA+EUP, which corresponds to the high weights obtained for BS and maternal age using LASSO regression.

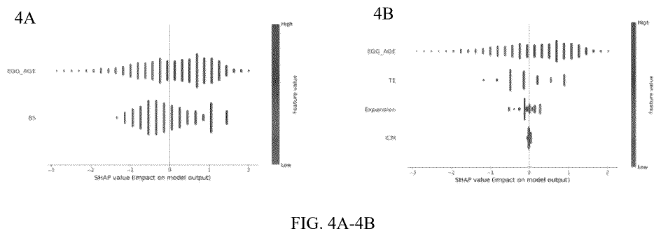

When looking at feature importance, we find that maternal age positively correlates with aneuploid, as well as BS (FIG. 4A-B). This is in agreement with previous literature for maternal age, and for BS, since lower scores are defined as higher quality embryos (32). When analyzing the individual components of the blastocyst grade, we see that changes in the TE grade had the largest impact on model performance, followed by the expansion grade, and then the ICM grade. This can point to some biological relevance since the cells biopsied and whose DNA is used for sequencing are from the TE.

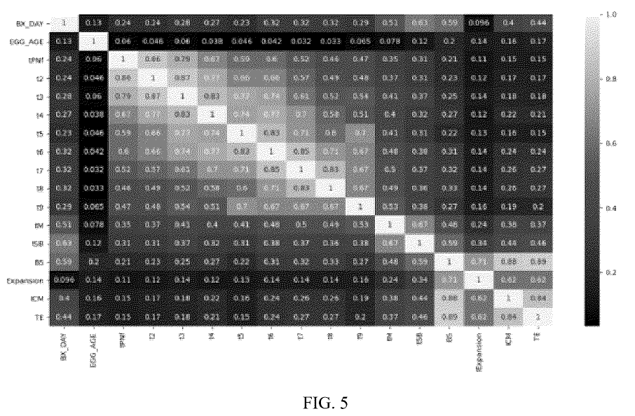

As an additional step to analyze feature importance, a univariate assessment of each morphological feature was performed in combination with egg age. This analysis supports TE grade being most predictive of ploidy, with an accuracy of 0.703 (95% CI: 0.684-0.722), followed by ICM grade 0.697 (95% CI: 0.684-0.710), and Expansion grade 0.692 (95% CI: 0.677-0.706) for the ANU vs. EUP classification. This trend holds for both CA vs. EUP (0.773-95% CI: 0.766-0.780, 0.754-95% CI: 0.747-0.760, and 0.743-95% CI: 0.733-0.753 for TE, ICM, and Expansion respectively) and CA vs. ANU+EUP (0.706-95% CI: 0.694-0.717, 0.694-95% CI: 0.683-0.706, and 0.682-95% CI: 0.673-0.691 for TE, ICM, and Expansion respectively). A high Pearson correlation between ICM and TE grades, 0.84, can explain the low feature weight of the ICM grade in the multivariable analysis (FIG. 5).

TABLE 2
Logistic regression - morphological importance: Maternal age, the three components of blastocyst grade
(ICM Grade, TE Grade, and Expansion Grade) and blastocyst score (BS) were analyzed using logistic
regression to determine feature importance. Logistic regression was applied to several data splits
however, it was observed that regardless of the split, Maternal age with TE Grade and Maternal age
with BS were consistently weighted higher indicating their importance for ploidy prediction.

| | | | | | | | 95% |
|---|---|---|---|---|---|---|---|
| | Maternal age | ICM | | Expansion | Blastocyst | | Confidence |
| | Feature | Grade | TE Grade | Grade | Score | Model | Interval |
| Model | Weight ** | Weight ** | Weight ** | Weight ** | Weight ** | Accuracy | Bounds |
| ANU vs. | 0.864 ± 0.009 | −0.058 ± 0.038 | −0.504 ± 0.036 | −0.17.2 ± 0.014 | . . . | 0.708 | 0.696-0.720 |
| EUP BG | | | | | | | |
| ANU vs. | 0.852 ± 0.01 | . . . | . . . | . . . | 0.705 ± 0.011 | 0.703 | 0.693-0.713 |
| EUP BS | | | | | | | |
| CA vs. | 1.227 ± 0.034 | −0.043 ± 0.031 | −0.670 ± 0.018 | −0.225 ± 0.044 | . . . | 0.770 | 0.756-0.785 |
| EUP BG | | | | | | | |
| CA vs. | 1.206 ± 0.036 | . . . | . . . | . . . | 0.910 ± 0.027 | 0.772 | 0.758-0.787 |
| EUP BS | | | | | | | |
| CA vs. SA + | 0.919 ± 0.013 | −0.021 ± 0.029 | −0.484 ± 0.017 | −0.155 ± 0.008 | — | 0.713 | 0.703-0.723 |
| EUP BG | | | | | | | |
| CA vs. SA + | 0.907 ± 0.013 | . . . | . . . | . . . | 0.649 ± 0.012 | 0.711 | 0.697-0.724 |
| EUP BS | | | | | | | |
| CA vs. SA + | . . . | . . . | . . . | . . . | . . . | 0.718 | 0.705-0.731 |
| EUP | | | | | | | |
| All Features | | | | | | | |

### Example 4

### Evaluation of Machine and Deep Learning Models for Ploidy Prediction

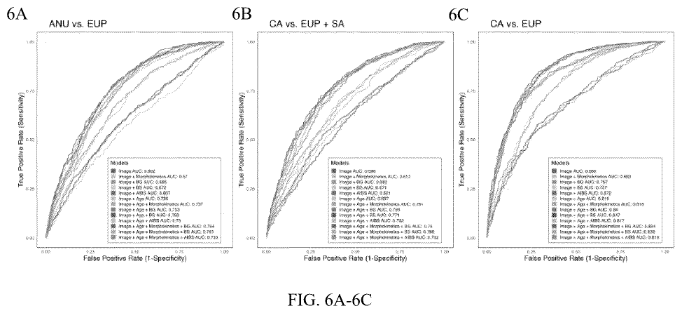

As logistic regression is a linear model, the subsequent step in the study was to understand how more complex and non-linear machine learning approaches, specifically XGBoost, k-NN, SVM, and Random Forest, would perform when predicting embryo ploidy. Each machine learning model was trained and tested using various combinations of clinical features across several classification tasks including ANU vs. EUP, CA vs. SA+EUP, and CA vs. EUP (FIG. 6A-C).

Across the three classification tasks, SVM and XGBoost generally performed best except for the Random Forest for the CA vs. EUP+SA task (Table 3). Amongst the four architectures, k-NN performed the worst. In the ANU vs. EUP task, the SVM using maternal age, morphokinetics, and BS demonstrated an accuracy of 70.5% (95% CI: 68.2-72.8%). For the CA vs. EUP+SA task, Random Forest reported an accuracy of 76.8% (95% CI: 74.6-78.9%) using maternal age, morphokinetics, and BS. Lastly, in the CA vs. EUP classification task, XGBoost and SVM shared the same performance of 77.6 (95% CI: 75.0-80.0%) using maternal age and BS. A review of the performance of the models with a single clinical feature, models 1 thru 5, indicates that maternal age alone is a strong predictor of ploidy status across all classification tasks. The addition of morphological assessments, either BG, BS, or AIBS, to maternal age generally improved model accuracies across the board in all three classification tasks. On the other hand, morphokinetic parameters in the ANU vs. EUP and CA vs. EUP tasks generally did not improve performance in alignment with findings from the regression analyses. However, in the CA vs. EUP+SA task, the addition of morphokinetic parameters to Random Forest models improved performance.

Next, STORK-A, a deep learning CNN based on a modified ResNet18 architecture, was used to extract features from static images of embryos at 110 hpi that were then concatenated with the previously used clinical features to predict ploidy. At a baseline, models trained using only images for the classification tasks ANU vs. EUP, CA vs. SA+EUP, and CA vs. EUP reported accuracies of 59.2% (95% CI: 56.7-61.6%), 61.1% (95% CI: 58.6-63.5%), and 64.0% (95% CI: 61.1-66.8%) respectively (Table 4). Similar to what was observed in the machine learning models, the addition of morphological assessments along with maternal age improved model accuracy in all three classification tasks. Again, morphokinetic parameters did not provide significant improvement to the models and in some cases decreased performance. The best performing models for the ANU vs. EUP (Acc: 69.3%-95% CI: 66.9-71.5%), CA vs. EUP+SA (Acc: 74.0%-95% CI: 71.7-76.1%), and CA vs. EUP (Acc: 77.6%-95% CI: 75.0-80.0%) classification tasks utilized images, maternal age, and morphokinetic parameters, morphological assessment (BG or BS). For the CA vs. EUP task, the model including image, maternal age, and BG performed similarly with an accuracy of 77.6% (95% CI: 75.1-80.1%) but this resulted in a trade-off in PPV and NPV.

Tested against the primary test set, STORK-A for ANU vs. EUP reported an accuracy of 69.3%. When the primary test aneuploids in the ANU vs. EUP classification task were stratified, it was observed that STORK-A correctly predicted 77.1% of CA embryos and correctly predicted 57.0% of SA embryos (Table 5). It is plausible that SA and EUP embryos share an overlap in morphology and morphokinetics, making it difficult for STORK-A to differentiate between the two classes. STORK-A for CA vs EUP+SA was poised to verify this overlap assumption and reported an accuracy of 74.0%. This classifier was able to identify 89.8% of all euploid embryos, 66.7% of SA embryos, and only 57.6% of CA (Table 6). Given these results, it is more likely that SA embryos share an overlap amongst both CA and EUP as the accuracy of the CA class decreased while EUP increased.

TABLE 3
Machine learning performance: Four machine learning architectures, XGBoost, k-NN, SVM, and
Random Forest were trained, validated, and tested for three classification tasks, ANU vs.
EUP, CA vs. SA + EUP, and CA vs. EUP. Various combinations of clinical features, including
maternal age, morphological assessment, including BS, BG, and AIBS, and morphokinetic parameters,
were used for input. Performance measurements include accuracy (Acc.), 95% CI, and PPV.

| | XGBoost | | | k-NN | | | SVM | | | Random Forest | | |
|---|---|---|---|---|---|---|---|---|---|---|---|---|
| | Acc. | 95% | PPV | Acc. | 95% | PPV | Acc. | 95% | PPV | Acc. | 95% | PPV |
| | % | CI | % | % | CI | % | % | CI | % | % | CI | % |
| ANU vs. EUP | | | | | | | | | | | | |
| 1. Age | 66.3 | 63.9- | 74.1 | 66.3 | 63.9- | 74.1 | 66.3 | 63.9- | 74.1 | 66.3 | 63.9- | 68.7 |
| | | 68.7 | | | 68.7 | | | 68.7 | | | 68.7 | |
| 2. Morphokinetics | 54.8 | 52.3- | 62.3 | 52.5 | 50.0- | 59.9 | 55.4 | 52.9- | 64.0 | 56.5 | 54.0- | 61.8 |
| | | 57.3 | | | 55.0 | | | 57.9 | | | 59.0 | |
| 3. BS | 62.7 | 60.2- | 74.0 | . . . | . . . | . . . | 62.7 | 60.2- | 74.0 | 62.7 | 60.2- | 74.0 |
| | | 65.1 | | | | | | 65.1 | | | 65.1 | |
| 4. BG | 63.2 | 60.8- | 72.2 | 61.4 | 58.9- | 72.6 | 61.0 | 58.5- | 71.1 | 62.4 | 59.9- | 74.5 |
| | | 65.6 | | | 63.8 | | | 63.4 | | | 64.8 | |
| 5. AIBS | 57.5 | 55.0- | 64.7 | 56.5 | 54.0- | 63.4 | 57.4 | 54.9- | 64.9 | 53.7 | 51.2- | 59.4 |
| | | 60.0 | | | 59.0 | | | 59.8 | | | 56.2 | |
| 6. Age + | 65.3 | 62.8- | 72.6 | 63.6 | 61.2- | 71.1 | 66.0 | 63.6- | 73.5 | 65.1 | 62.7- | 70.0 |
| Morphokinetics | | 67.6 | | | 66.0 | | | 68.4 | | | 67.5 | |
| 7. Age + BS | 68.5 | 66.1- | 78.0 | 69.7 | 67.4- | 77.5 | 70.2 | 67.8- | 77.8 | 69.1 | 66.8- | 77.8 |
| | | 70.8 | | | 72.0 | | | 72.4 | | | 71.4 | |
| 8. Age + BG | 68.5 | 66.1- | 77.3 | 66.4 | 64.0- | 75.6 | 69.0 | 66.6- | 76.9 | 67.3 | 64.9- | 75.8 |
| | | 70.8 | | | 68.8 | | | 71.3 | | | 69.7 | |
| 9. Age + AIBS | 66.8 | 64.4- | 74.6 | 63.4 | 61.0- | 70.6 | 67.7 | 65.3 | 70.0 | 61.2 | 58.7- | 66.4 |
| | | 69.2 | | | 65.8 | | | | | | 63.7 | |
| 10. Age + | 68.1 | 65.7- | 75.4 | 63.1 | 60.6- | 72.2 | 70.5 | 68.2- | 78.6 | 67.5 | 65.1- | 73.2 |
| Morphokinetics + BS | | 70.4 | | | 65.5 | | | 72.8 | | | 69.8 | |
| 11. Age + | 67.4 | 65.0- | 73.1 | 64.3 | 61.9- | 72.1 | 69.2 | 66.8- | 77.9 | 68.6 | 66.2- | 70.9 |
| Morphokinetics + BG | | 69.7 | | | 66.7 | | | 71.5 | | | 70.9 | |
| 12. Age + | 65.7 | 63.3- | 71.9 | 61.7 | 59.2- | 68.5 | 66.2 | 63.8- | 73.9 | 66.8 | 64.4- | 71.4 |
| Morphokinetics + | | 68.1 | | | 64.1 | | | 68.5 | | | 69.2 | |
| AIBS | | | | | | | | | | | | |
| CA vs. EUP + SA | | | | | | | | | | | | |
| 1. Age | 68.1 | 65.7- | 46.7 | . . . | . . . | . . . | 71.1 | 68.8- | 50.2 | 68.1 | 65.7- | 46.7 |
| | | 70.4 | | | | | | 73.4 | | | 70.4 | |
| 2. Morphokinetics | 60.6 | 58.1- | 33.3 | 53.6 | 51.1- | 31.8 | 59.5 | 57.0- | 37.4 | 68.9 | 66.6- | 42.1 |
| | | 63.0 | | | 56.1 | | | 61.9 | | | 71.2 | |
| 3. BS | 66.0 | 63.6- | 44.1 | . . . | . . . | . . . | 66.0 | 63.6- | 44.1 | 66.0 | 63.6- | 44.1 |
| | | 68.4 | | | | | | 68.4 | | | 68.4 | |
| 4. BG | 65.3 | 62.8- | 43.2 | . . . | . . . | . . . | 64.6 | 62.2- | 42.8 | 65.2 | 62.8- | 43.4 |
| | | 67.6 | | | | | | 67.0 | | | 67.6 | |
| 5. AIBS | 57.4 | 54.9- | 36.1 | 55.9 | 53.4- | 34.1 | 59.0 | 56.5- | 38.5 | 60.8 | 58.4- | 31.9 |
| | | 59.8 | | | 58.4 | | | 61.5 | | | 63.3 | |
| 6. Age + | 69.9 | 67.6- | 48.2 | 64.1 | 61.7- | 42.1 | 70.0 | 67.7- | 48.8 | 75.2 | 73.0- | 58.9 |
| Morphokinetics | | 72.2 | | | 66.5 | | | 72.3 | | | 77.3 | |
| 7. Age + BS | 71.6 | 69.3- | 50.7 | 71.6 | 69.3- | 50.8 | 72.5 | 70.2- | 52.0 | 71.4 | 69.1- | 50.5 |
| | | 73.8 | | | 73.8 | | | 74.7 | | | 73.6 | |
| 8. Age + BG | 71.3 | 69.0- | 50.4 | 69.6 | 67.3- | 48.4 | 72.2 | 69.9- | 51.6 | 70.5 | 68.2- | 49.4 |
| | | 73.6 | | | 71.9 | | | 74.4 | | | 72.8 | |
| 9. Age + AIBS | 70.2 | 67.9- | 49.0 | 65.7 | 63.3- | 43.9 | 71.0 | 68.7- | 50.0 | 68.3 | 65.9- | 45.1 |
| | | 72.5 | | | 68.1 | | | 73.2 | | | 70.6 | |
| 10. Age + | 72.2 | 69.9- | 51.8 | 66.2 | 63.8- | 44.3 | 72.8 | 70.5- | 52.4 | 76.8 | 74.6- | 61.8 |
| Morphokinetics + BS | | 74.4 | | | 68.6 | | | 75.0 | | | 78.9 | |
| 11. Age + | 72.0 | 69.7- | 51.4 | 65.0 | 62.5- | 42.9 | 72.0 | 69.7- | 51.3 | 75.2 | 73.0- | 57.9 |
| Morphokinetics + BG | | 74.2 | | | 67.3 | | | 74.2 | | | 77.3 | |
| 12. Age + | 71.0 | 68.7- | 50.0 | 65.0 | 62.6- | 43.3 | 70.8 | 68.5- | 49.8 | 74.8 | 72.6- | 58.0 |
| Morphokinetics + | | 73.2 | | | 67.4 | | | 73.1 | | | 76.9 | |
| AIBS | | | | | | | | | | | | |
| CA vs. EUP | | | | | | | | | | | | |
| 1. Age | 74.1 | 71.5- | 67.4 | 74.1 | 71.5- | 67.4 | 74.1 | 71.4- | 67.2 | 74.1 | 71.4- | 67.2 |
| | | 76.7 | | | 76.7 | | | 76.6 | | | 76.6 | |
| 2. Morphokinetics | 59.7 | 56.7- | 50.2 | 53.6 | 50.6- | 44.1 | 61.3 | 58.4- | 51.9 | 59.2 | 56.2- | 49.3 |
| | | 62.6 | | | 56.6 | | | 64.2 | | | 62.1 | |
| 3. BS | 72.3 | 69.5- | 65.8 | 72.3 | 69.5- | 65.8 | 72.3 | 69.5- | 65.8 | 72.3 | 69.5- | 65.8 |
| | | 74.9 | | | 74.9 | | | 74.9 | | | 74.9 | |
| 4. BG | 70.6 | 67.9- | 63.2 | 70.8 | 68.1- | 63.4 | 71.2 | 68.4- | 63.5 | 71.6 | 68.9- | 65.4 |
| | | 73.3 | | | 73.5 | | | 73.8 | | | 74.3 | |
| 5. AIBS | 63.3 | 60.4- | 53.6 | 60.4 | 57.5 | 63.3 | 63.6 | 60.7- | 54.1 | 57.4 | 54.4- | 47.3 |
| | | 66.1 | | | | | | 66.5 | | | 60.3 | |
| 6. Age + | 72.6 | 69.9- | 67.0 | 68.8 | 65.9- | 60.2 | 73.7 | 71.0- | 67.0 | 72.6 | 69.9- | 68.6 |
| Morphokinetics | | 75.2 | | | 71.5 | | | 76.3 | | | 75.2 | |
| 7. Age + BS | 77.6 | 75.0- | 73.0 | 77.4 | 74.8- | 73.2 | 77.6 | 75.0- | 73.1 | 77.0 | 74.4- | 72.9 |
| | | 80.0 | | | 79.8 | | | 80.0 | | | 79.3 | |
| 8. Age + BG | 76.8 | 74.2- | 71.9 | 76.7 | 74.1- | 71.3 | 76.9 | 74.3- | 71.3 | 76.6 | 74.0- | 71.8 |
| | | 79.3 | | | 79.1 | | | 79.4 | | | 79.0 | |
| 9. Age + AIBS | 75.5 | 72.9- | 69.2 | 72.6 | 69.9- | 65.3 | 75.2 | 72.6- | 69.1 | 68.9 | 66.0- | 61.6 |
| | | 78.0 | | | 75.2 | | | 77.7 | | | 71.6 | |
| 10. Age + | 76.5 | 73.9- | 71.5 | 75.0 | 72.3- | 67.7 | 77.3 | 74.7- | 72.1 | 76.2 | 73.6- | 73.0 |
| Morphokinetics + BS | | 78.9 | | | 77.5 | | | 79.7 | | | 78.7 | |
| 11. Age + | 77.2 | 74.6- | 72.5 | 74.1 | 71.5- | 67.0 | 77.2 | 74.6- | 71.9 | 75.9 | 73.2- | 71.5 |
| Morphokinetics + BG | | 79.6 | | | 76.7 | | | 79.6 | | | 78.3 | |
| 12. Age + | 73.1 | 70.4- | 67.5 | 70.4 | 67.6- | 61.8 | 75.6 | 73.0- | 69.6 | 74.1 | 71.4- | 70.4 |
| Morphokinetics + | | 75.7 | | | 73.0 | | | 78.1 | | | 76.6 | |
| AIBS | | | | | | | | | | | | |

TABLE 4
Deep learning performance: A modified ResNet18 architecture was trained, validated, and tested for three classification
tasks, ANU vs. EUP, CA vs. SA + EUP, and CA vs. EUP. Combinations of the clinical feature and images were
used to develop the models. Performance measurements include accuracy, 95% CI, PPV, and NPV. The test data set
and its predictions were then stratified into demographics based on age and day of blastocyst formation.

| | Performance | | | | Stratified test data accuracy | | | | | |
|---|---|---|---|---|---|---|---|---|---|---|
| | Model | | | | | 35 < | 37 < | | | |
| | Accuracy | 95% | PPV | NPV | Age ≤ | Age ≤ | Age ≤ | Age > | | |
| | % | CI | % | % | 35 | 37 | 39 | 39 | Day 5 | Day 6 |
| ANU vs. EUP | | | | | | | | | | |
| 1. Image | 59.2 | 56.7-61.6 | 63.7 | 52.3 | 52.1 | 56.3 | 62.5 | 67.1 | 55.7 | 61.3 |
| 2. Image + | 58.3 | 55.8-60.8 | 60.3 | 52.1 | 45.2 | 52.4 | 62.2 | 75.6 | 49.8 | 63.6 |
| Morphokinetics | | | | | | | | | | |
| 3. Image + BG | 63.9 | 61.5-66.3 | 71.2 | 56.6 | 61.2 | 61.4 | 63.0 | 70.2 | 57.6 | 67.8 |
| 4. Image + BS | 62.2 | 59.7-64.6 | 69.2 | 54.9 | 60.4 | 57.6 | 60.6 | 69.7 | 54.7 | 66.8 |
| 5. Image + AIBS | 59.3 | 56.8-61.7 | 62.9 | 52.7 | 52.3 | 55.3 | 60.9 | 69.7 | 53.7 | 62.7 |
| 6. Image + Age | 67.8 | 65.5-70.2 | 73.4 | 61.3 | 63.7 | 55.3 | 64.9 | 85.9 | 66.6 | 68.6 |
| 7. Image + Age + | 67.8 | 65.4-70.1 | 73.5 | 61.2 | 63.5 | 56.3 | 64.3 | 85.6 | 66.0 | 68.8 |
| Morphokinetics | | | | | | | | | | |
| 8. Image + Age + BG | 68.7 | 66.3-71.0 | 71.5 | 64.4 | 63.5 | 57.2 | 67.3 | 85.6 | 65.2 | 70.8 |
| 9. Image + Age + BS | 69.0 | 66.6-71.3 | 75.1 | 62.3 | 65.1 | 59.2 | 64.9 | 85.6 | 65.4 | 71.2 |
| 10. Image + Age + AIBS | 66.5 | 64.1-68.8 | 71.2 | 60.4 | 61.8 | 54.7 | 63.8 | 84.3 | 65.0 | 67.4 |
| 11. Image + Age + BG + | 68.9 | 66.5-71.2 | 74.2 | 62.6 | 63.9 | 60.1 | 66.0 | 84.8 | 66.7 | 70.2 |
| Morphokinetics | | | | | | | | | | |
| 12. Image + Age + BS + | 69.3 | 66.9-71.5 | 76.1 | 62.1 | 63.9 | 62.1 | 65.7 | 85.1 | 66.2 | 71.1 |
| Morphokinetics | | | | | | | | | | |
| 13. Image + Age + AIBS + | 67.0 | 64.6-69.3 | 71.66 | 61.1 | 64.5 | 51.4 | 64.1 | 85.3 | 65.2 | 68.1 |
| Morphokinetics | | | | | | | | | | |
| CA vs. EUP + SA | | | | | | | | | | |
| 1. Image | 61.1 | 58.6-63.5 | 37.0 | 76.0 | 67.1 | 60.2 | 61.0 | 55.0 | 75.9 | 52.2 |
| 2. Image + | 61.5 | 59.0-63.9 | 37.1 | 75.7 | 68.4 | 63.1 | 59.4 | 54.0 | 76.9 | 52.2 |
| Morphokinetics | | | | | | | | | | |
| 3. Image + BG | 67.1 | 64.7-69.4 | 44.5 | 79.7 | 73.1 | 67.8 | 63.4 | 63.0 | 73.3 | 63.3 |
| 4. Image + BS | 68.2 | 65.8-70.5 | 45.5 | 78.6 | 75.6 | 69.9 | 64.2 | 62.0 | 76.2 | 63.3 |
| 5. Image + AIBS | 64.4 | 62.0-66.8 | 38.8 | 75.1 | 71.8 | 69.6 | 62.6 | 53.2 | 76.7 | 57.1 |
| 6. Image + Age | 70.5 | 68.1-72.7 | 48.8 | 76.5 | 85.8 | 75.2 | 61.5 | 57.3 | 77.2 | 66.4 |
| 7. Image + Age + | 71.6 | 69.3-73.8 | 50.8 | 84.1 | 87.6 | 79.9 | 57.8 | 59.1 | 76.4 | 68.7 |
| Morphokinetics | | | | | | | | | | |
| 8. Image + Age + BG | 72.2 | 69.9-74.4 | 51.7 | 83.5 | 87.3 | 77.0 | 60.5 | 62.0 | 77.1 | 69.3 |
| 9. Image + Age + BS | 73.2 | 70.9-75.4 | 53.2 | 83.4 | 86.2 | 75.8 | 62.6 | 66.1 | 79.3 | 69.5 |
| 10. Image + Age + AIBS | 72.0 | 69.7-74.2 | 51.4 | 83.0 | 86.2 | 79.1 | 59.2 | 61.7 | 78.8 | 67.9 |
| 11. Image + Age + BG + | 74.0 | 71.7-76.1 | 54.9 | 82.3 | 87.6 | 77.3 | 62.6 | 66.3 | 78.8 | 71.1 |
| Morphokinetics | | | | | | | | | | |
| 12. Image + Age + BS + | 71.6 | 69.3-73.8 | 50.8 | 83.9 | 87.1 | 72.9 | 61.5 | 62.2 | 77.4 | 68.1 |
| Morphokinetics | | | | | | | | | | |
| 13. Image + Age + AIBS + | 73.0 | 70.7-75.2 | 53.3 | 81.4 | 86.9 | 80.2 | 62.1 | 61.2 | 77.6 | 70.2 |
| Morphokinetics | | | | | | | | | | |
| CA vs. EUP | | | | | | | | | | |
| 1. Image | 64.0 | 61.1-66.8 | 55.2 | 70.7 | 67.6 | 69.3 | 58.5 | 60.9 | 74.4 | 58.1 |
| 2. Image + | 62.6 | 59.7-65.4 | 54.4 | 66.9 | 70.6 | 68.0 | 55.1 | 56.1 | 76.6 | 54.4 |
| Morphokinetics | | | | | | | | | | |
| 3. Image + BG | 71.5 | 68.7-74.1 | 67.0 | 73.9 | 75.5 | 73.8 | 69.1 | 67.3 | 77.1 | 68.2 |
| 4. Image + BS | 70.6 | 67.9-73.3 | 64.5 | 74.5 | 74.5 | 71.1 | 69.4 | 67.0 | 76.1 | 67.5 |
| 5. Image + AIBS | 63.3 | 60.4-66.1 | 55.1 | 68.1 | 70.6 | 70.2 | 60.0 | 52.7 | 76.1 | 55.9 |
| 6. Image + Age | 75.0 | 72.4-77.6 | 68.1 | 80.2 | 87.0 | 72.9 | 58.9 | 77.9 | 81.0 | 71.6 |
| 7. Image + Age + | 75.3 | 72.7-77.8 | 69.3 | 79.5 | 87.3 | 72.0 | 61.5 | 76.9 | 79.6 | 72.9 |
| Morphokinetics | | | | | | | | | | |
| 8. Image + Age + BG | 77.6 | 75.1-80.1 | 72.5 | 81.1 | 86.4 | 73.3 | 69.1 | 78.9 | 80.3 | 76.1 |
| 9. Image + Age + BS | 77.4 | 74.8-79.8 | 70.0 | 83.3 | 87.6 | 74.7 | 67.9 | 76.5 | 79.6 | 76.1 |
| 10. Image + Age + AIBS | 75.9 | 73.2-78.3 | 71.3 | 78.7 | 87.0 | 73.8 | 64.2 | 75.5 | 82.8 | 71.9 |
| 11. Image + Age + BG + | 77.0 | 74.4-79.5 | 71.3 | 81.0 | 87.0 | 73.3 | 67.5 | 77.2 | 79.6 | 76.1 |
| Morphokinetics | | | | | | | | | | |
| 12. Image + Age + BS + | 77.6 | 75.0-80.0 | 76.7 | 78.0 | 87.6 | 73.8 | 69.4 | 76.5 | 81.3 | 75.4 |
| Morphokinetics | | | | | | | | | | |
| 13. Image + Age + AIBS + | 75.0 | 72.4-77.6 | 69.4 | 78.9 | 87.3 | 68.0 | 64.2 | 76.5 | 82.3 | 70.9 |
| Morphokinetics | | | | | | | | | | |

TABLE 5
STORK-A confusion matrix ANU vs. EUP - image, age, morphokinetics, and BS.

| | Specificity: 71.5% | Sensitivity: 67.6% | |
|---|---|---|---|
| | Ground Truth | Ground Truth | |
| | Aneuploid | Euploid | Accuracy: 69% |
| Predicted Aneuploid | 603 | 189 | 792 |
| Predicted Euploid | 289 | 474 | 763 |
| | 892 | 663 | 1555 |
| | Single | Complex | Euploid |
| | Aneuploid (Sa) | Aneuploid (Ca) | (Eup) |
| Total Samples | 421 | 471 | 663 |
| Percent Correct | 57.0% | 77.1% | 71.5% |

TABLE 6
STORK-A confusion matrix CA vs. EUP +
SA - image, age, morphokinetics, and BG.

| | Specificity: 80.1% | Sensitivity: 57.6% | |
|---|---|---|---|
| | Ground Truth | Ground Truth Euploid + | |
| | Complex Aneuploid | Single Aneuploid | Accuracy: 74.0% |
| Predicted | 260 | 214 | 474 |
| Complex | | | |
| Aneuploid | | | |
| Predicted | 191 | 890 | 1081 |
| Euploid + | | | |
| Single | | | |
| Aneuploid | | | |
| | 451 | 1104 | 1555 |
| | Single | Complex | Euploid |
| | Aneuploid (SA) | Aneuploid (CA) | (EUP) |
| Total | 441 | 451 | 663 |
| Samples | | | |
| Percent | 66.7% | 57.6% | 89.8% |
| Correct | | | |

TABLE 7
STORK-A confusion matrix CA vs. EUP -
image, age, morphokinetics, and BS.

| | Specificity: 86.7% | Sensitivity: 64.1% | |
|---|---|---|---|
| | Ground Truth | Ground Truth | |
| | Complex Aneuploid | Euploid | Accuracy: 77.6% |
| Predicted | 289 | 88 | 377 |
| Complex | | | |
| Aneuploid | | | |
| Predicted | 162 | 575 | 737 |
| Euploid | | | |
| | 451 | 663 | 1114 |

### Example 5

### Artificial Intelligence Blastocyst Score (AIBS)

Our results indicate the utility of blastocyst morphology assessment, both BG and BS, for ploidy prediction in both machine and deep learning models. However, morphology assessment is subject to observer variability and bias. To circumvent this issue, blastocyst scores were predicted utilizing deep learning and regression with embryologist-derived blastocyst scores as ground truth labels. The model reported a mean squared error (MSE) of 16.3 and a Pearson Correlation coefficient of 0.65 for AIBS. The AIBS for each embryo in the primary dataset was then used as input for all machine and deep learning classification tasks. In general, AIBS underperformed compared to embryologist-derived BS and BG. However, AIBS does offer an improvement over age alone in machine learning models for all three classification tasks, and image and age alone in deep learning models for CA vs EUP+SA and CA vs EUP classification tasks.

### Example 6

### Post-Hoc Analysis Based on Age

Embryos in the primary test set and their predictions were further categorized by age groups, Age <35, 35<Age <37, 37<Age <39, and Age >39, to examine whether there were differences in the model's ability to predict ploidy status based on age. Of interest were the embryos patients younger than 36 and patients older than 39, as several studies have concluded that not all patients need or should utilize PGT-A. Within the youngest age group of the primary test set, STORK-A for ANU vs. EUP using an image, age, morphokinetic parameters, and BS correctly classified 63·% of embryos, with a specificity of 93.5% and sensitivity of 12.0%. While the model can sufficiently identify euploid embryos, it struggles to correctly identify aneuploids and suffers from false negatives. Therefore STORK-A can be beneficial as a screening tool to identify euploids without being hindered by a large number of false positives in embryos associated with maternal age younger than 36. For the embryos with maternal age older than 39, the same STORK-A model correctly predicted 85.1% of embryos with a specificity of 5.4% and sensitivity of 98.5%. The severe class imbalance of aneuploids and euploids in this age group of embryos is the cause for the stark differences in sensitivity and specificity. Nonetheless, the high sensitivity for this age group would be useful for identifying aneuploid embryos without generating many false negative predictions.

Given these findings, an optimal threshold that maximizes the sum of the specificity and sensitivity for embryos with maternal age between 37 and 42 was assessed (Table 8). For embryos with maternal of 37 years old, the performance using optimal threshold only slightly deviated from the baseline 50/50 threshold, and therefore did not significantly improve performance. For embryos with maternal ages between 38 to 42, we identified a trend that indicates that optimal thresholds can be useful depending on an embryologist's intentions. That is, whether it is favorable to deselect as many aneuploids as possible but also risk the deselection of some euploids, a higher sensitivity, or confidently identifying euploid embryos but risking the inclusion of some aneuploids. For example, in embryos with maternal age of 43, a 50/50 decision threshold has an accuracy of 90.9%, with a high sensitivity of 98.9%. However, this comes at the cost of predicting only one embryo as euploid resulting in a low specificity of 11.1%. By applying an optimal decision threshold of 0.350 to maximize the sum of specificity and sensitivity, the overall accuracy drops to 70.7% but in this instance, there is a benefit as this incurs an increase in the specificity which reaches 66.7% by correctly classifying 6 of 9 euploids but misclassifying 26 aneuploids as euploid.

TABLE 8
Optimal thresholds for ANU vs. EUP classifier.
50/50 Threshold for Age 37

| | Ground | Ground | |
|---|---|---|---|
| | Truth | Truth | |
| | Aneuploid | Euploid | |
| Predicted | 34 | 17 | 51 |
| Aneuploid | | | |
| Predicted | 23 | 40 | 63 |
| Euploid | | | |
| | 57 | 57 | 114 |

Overall Accuracy: 64.9%
Specificity: 70.2%
Sensitivity: 59.6%

Optimal Cutoff Threshold for Age 37: 0.503

| | Ground | Ground | |
|---|---|---|---|
| | Truth | Truth | |
| | Aneuploid | Euploid | |
| Predicted | 35 | 18 | 53 |
| Aneuploid | | | |
| Predicted | 22 | 39 | 61 |
| Euploid | | | |
| | 57 | 57 | 114 |

Overall Accuracy: 64.9%
Specificity: 68.4%
Sensitivity: 61.4%

50/50 Threshold for Age 38

| | Ground | Ground | |
|---|---|---|---|
| | Truth | Truth | |
| | Aneuploid | Euploid | |
| Predicted | 32 | 20 | 52 |
| Aneuploid | | | |
| Predicted | 17 | 29 | 46 |
| Euploid | | | |
| | 49 | 49 | 98 |

Overall Accuracy: 62.2%
Specificity: 59.2%
Sensitivity: 65.3%

Optimal Cutoff Threshold for Age 38: 0.377

| | Ground | Ground | |
|---|---|---|---|
| | Truth | Truth | |
| | Aneuploid | Euploid | |
| Predicted | 25 | 4 | 29 |
| Aneuploid | | | |
| Predicted | 24 | 45 | 69 |
| Euploid | | | |
| | 49 | 49 | 98 |

Overall Accuracy: 71.4%
Specificity: 94.8%
Sensitivity: 51.0%

50/50 Threshold for Age 39

| | Ground | Ground | |
|---|---|---|---|
| | Truth | Truth | |
| | Aneuploid | Euploid | |
| Predicted | 49 | 23 | 72 |
| Aneuploid | | | |
| Predicted | 21 | 23 | 44 |
| Euploid | | | |
| | 70 | 46 | 116 |

Overall Accuracy: 62.1%
Specificity: 50.0%
Sensitivity: 70.0%

Optimal Cutoff Threshold for Age 39: 0.440

| | Ground | Ground | |
|---|---|---|---|
| | Truth | Truth | |
| | Aneuploid | Euploid | |
| Predicted | 41 | 13 | 54 |
| Aneuploid | | | |
| Predicted | 29 | 33 | 62 |
| Euploid | | | |
| | 70 | 46 | 116 |

Overall Accuracy: 63.8%
Specificity: 71.7%
Sensitivity: 58.6%

50/50 Threshold for Age 40

| | Ground | Ground | |
|---|---|---|---|
| | Truth | Truth | |
| | Aneuploid | Euploid | |
| Predicted | 101 | 35 | 136 |
| Aneuploid | | | |
| Predicted | 12 | 11 | 23 |
| Euploid | | | |
| | 113 | 46 | 159 |

Overall Accuracy: 70.4%
Specificity: 23.9%
Sensitivity: 89.4%

Optimal Cutoff Threshold for Age 40: 0.431

| | Ground | Ground | |
|---|---|---|---|
| | Truth | Truth | |
| | Aneuploid | Euploid | |
| Predicted | 69 | 13 | 82 |
| Aneuploid | | | |
| Predicted | 44 | 33 | 77 |
| Euploid | | | |
| | 113 | 46 | 159 |

Overall Accuracy: 64.2%
Specificity: 71.7%
Sensitivity: 61.1%

50/50 Threshold for Age 41

| | Ground | Ground | |
|---|---|---|---|
| | Truth | Truth | |
| | Aneuploid | Euploid | |
| Predicted | 96 | 31 | 127 |
| Aneuploid | | | |
| Predicted | 3 | 2 | 5 |
| Euploid | | | |
| | 99 | 33 | 132 |

Overall Accuracy: 74.2%
Specificity: 6.1%
Sensitivity: 97.0%

Optimal Cutoff Threshold for Age 41: 0.306

| | Ground | Ground | |
|---|---|---|---|
| | Truth | Truth | |
| | Aneuploid | Euploid | |
| Predicted | 55 | 5 | 60 |
| Aneuploid | | | |
| Predicted | 44 | 28 | 72 |
| Euploid | | | |
| | 99 | 33 | 132 |

Overall Accuracy: 62.9%
Specificity: 84.8%
Sensitivity: 55.6%

50/50 Threshold for Age 42

| | Ground | Ground | |
|---|---|---|---|
| | Truth | Truth | |
| | Aneuploid | Euploid | |
| Predicted | 89 | 8 | 97 |
| Aneuploid | | | |
| Predicted | 1 | 1 | 2 |
| Euploid | | | |
| | 90 | 9 | 99 |

Overall Accuracy: 90.9%
Specificity: 11.1%
Sensitivity: 98.9%

Optimal Cutoff Threshold for Age 42: 0.350

| | Ground | Ground | |
|---|---|---|---|
| | Truth | Truth | |
| | Aneuploid | Euploid | |
| Predicted | 64 | 3 | 67 |
| Aneuploid | | | |
| Predicted | 26 | 6 | 32 |
| Euploid | | | |
| | 90 | 9 | 99 |

Overall Accuracy: 70.7%
Specificity: 66.7%
Sensitivity: 71.1%

### Example 7

### Post-Hoc Analysis of Fetal Heart and Live Birth Outcomes

A downstream event to ploidy prediction is the presence of a fetal heart and live birth. Table 9 shows the fetal heart rate and live birth rate of 242 transferred embryos that were classified as euploid by PGT-A. The ability of STORK-A for ANU vs. EUP to correctly predict euploid embryos was compared to the fetal heart and live birth rates of embryos determined to be euploid by PGT-A. Of the 242 embryos, STORK-A predicted 166 (68.6%) embryos to be euploid. Of these 166, 56.02% resulted in a fetal heart, which is similar to the rate established by PGT-A at 56.61%. When investigating the live birth rates, embryos predicted to be euploid by STORK-A exhibited a live birth rate of 48.19%, again comparable to the rate observed by PGT-A of 48.76%. For patients 37 and younger, STORK-A demonstrates an ability to correctly predict embryos that will result in fetal hearts and live births.

TABLE 9
Fetal heart and live birth outcomes: 242 transferred embryos
labeled as euploid by PGT-A with known fetal heart and
live birth outcomes (global) were compared to embryos
predicted as aneuploid and euploid by STORK-A.

| | | | Fetal heart | | Live birth | |
|---|---|---|---|---|---|---|
| Age group | Type | Count | outcomes | | outcomes | |
| Overall | Global | 242 | 137 | (57%) | 118 | (49%) |
| | Predicted | 166 | 93 | (56%) | 80 | (48%) |
| | Euploid | | | | | |
| | Predicted | 76 | 44 | (58%) | 38 | (50%) |
| | Aneuploid | | | | | |
| Age ≤35 | Global | 86 | 51 | (59%) | 44 | (51%) |
| | Predicted | 83 | 51 | (61%) | 44 | (53%) |
| | Euploid | | | | | |
| | Predicted | 3 | 0 | (0%) | 0 | (0%) |
| | Aneuploid | | | | | |
| 35 < Age ≤ 37 | Global | 67 | 33 | (49%) | 28 | (42%) |
| | Predicted | 52 | 25 | (48%) | 21 | (40%) |
| | Euploid | | | | | |
| | Predicted | 15 | 8 | (53%) | 7 | (47%) |
| | Aneuploid | | | | | |
| 37 < Age ≤ 39 | Global | 56 | 33 | (59%) | 31 | (55%) |
| | Predicted | 29 | 15 | (52%) | 14 | (48%) |
| | Euploid | | | | | |
| | Predicted | 27 | 18 | (67%) | 17 | (63%) |
| | Aneuploid | | | | | |
| Age >39 | Global | 33 | 20 | (61%) | 15 | (45%) |
| | Predicted | 2 | 2 | (100%) | 1 | (50%) |
| | Euploid | | | | | |
| | Predicted | 31 | 18 | (58%) | 14 | (45%) |
| | Aneuploid | | | | | |

### Example 8

### Generalizability and Robustness of STORK-A

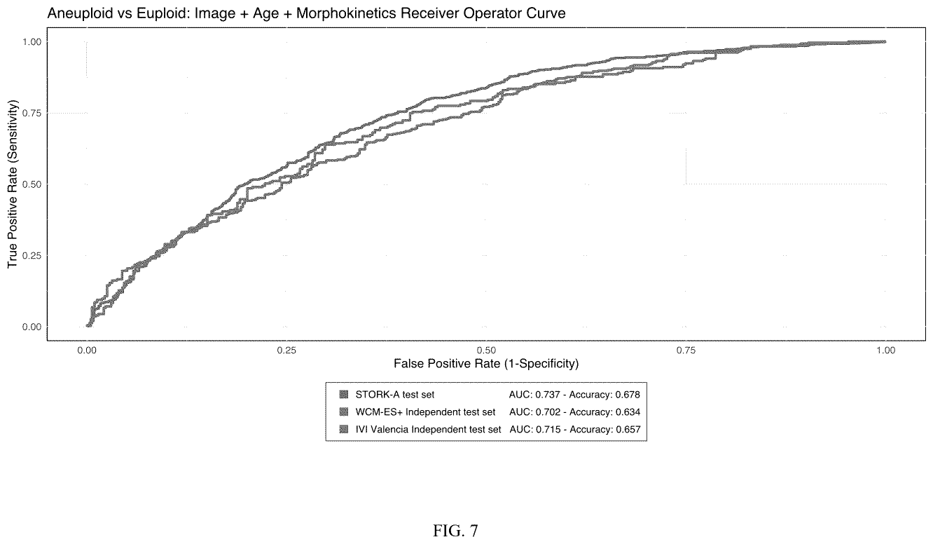

To test the robustness and generalizability of STORK-A, performance metrics from the primary test set were compared to those of two independent and external test sets. The first independent dataset is from the WCM Center of Reproduction and included images captured using the EmbryoScope+® (WCM-ES+). This data included 841 embryos along with maternal age, morphokinetic parameters, and morphological assessments (BG and BS). The second independent dataset is from IVI Valencia, Spain, and included images from 554 embryos captured using the original EmbryoScope®. The clinical information available included maternal age and morphokinetics. The trained STORK-A ANU vs. EUP classifier that utilizes images, morphokinetic parameters, and maternal age was tested on the WCM-ES+ and IVI Valencia test datasets. STORK-A produced accuracies of 63.4% (AUC=0.702) and 65.7 (AUC=0.715) respectively (FIG. 7). Compared to the accuracy of the primary test set, 67.8% (AUC=0.737), we see that STORK-A was able to maintain generalizability against the two external test sets. STORK-A for CA vs EUP+SA where image, maternal age, morphokinetics parameters, and BG were utilized as inputs was tested on the WCM-ES+ test data and resulted in an accuracy of 74.7% (AUC=0.781), similar to the accuracy on of the primary test set which was 74.0% (AUC=0.760).

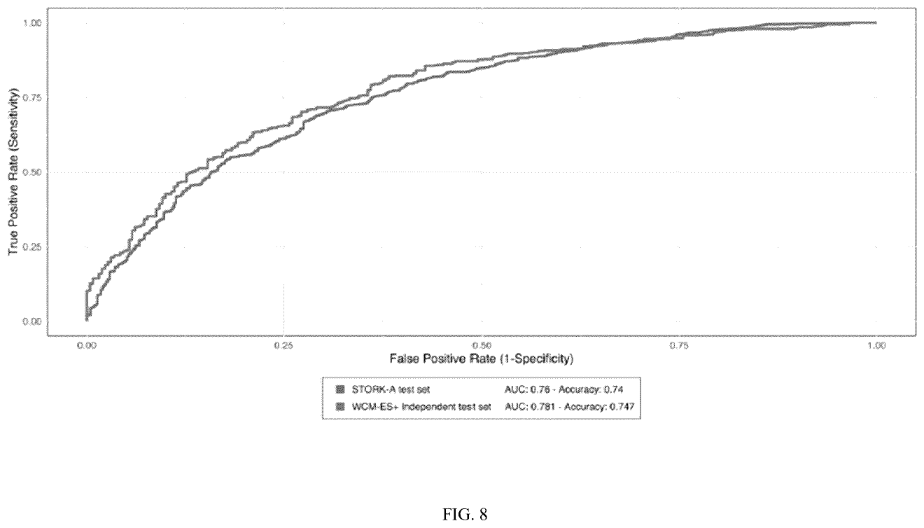

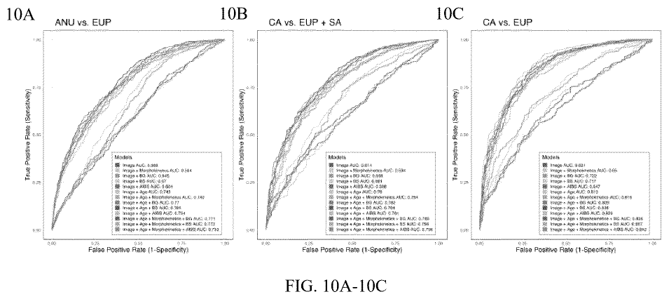

Performance of performance of CA vs EUP+SA with image, maternal age, morphokinetics, and performance of ANU vs EUP ith image and maternal age are shown in FIG. 8 and FIG. 9, respectively. Receiver operator curves for STORK-A classification tasks and models are shown in FIG. 10A-C. Model performance is summarized in Tables 10-14.

TABLE 10
Machine learning model performance (samples with complete data only*).

| | XGBoost | | | k-NN | | | SVM | | | Random Forest | | |
|---|---|---|---|---|---|---|---|---|---|---|---|---|
| | Acc. | 95% | PPV | Acc. | 95% | PPV | Acc. | 95% | PPV | Acc. | 95% | PPV |
| | % | CI | % | % | CI | % | % | CI | % | % | CI | % |
| ANU vs. EUP | | | | | | | | | | | | |
| 1. Age | 65.7 | 62.9-68.4 | 72.9 | 65.7 | 62.9-68.4 | 72.9 | 65.7 | 62.9-68.4 | 72.9 | 65.7 | 62.9-68.4 | 72.9 |
| 2. Morphokinetics | 53.4 | 50.5-56.2 | 57.1 | 52.1 | 49.2-55.0 | 56.1 | 57.2 | 54.3-60.0 | 61.9 | 52.8 | 49.9-55.6 | 55.5 |
| 3. BS | 62.4 | 59.6-65.2 | 75.6 | 62.4 | 59.6-65.2 | 75.6 | 62.4 | 59.6-65.2 | 75.6 | 62.4 | 59.6-65.2 | 75.6 |
| 4. BG | 63.0 | 60.2-65.8 | 67.8 | 60.2 | 57.3-62.9 | 65.3 | 61.7 | 58.9-64.5 | 66.1 | 61.3 | 58.5-64.1 | 69.8 |
| 5. AIBS | 58.3 | 55.4-61.1 | 59.5 | 58.2 | 52.6-58.3 | 58.1 | 58.2 | 55.4-61.0 | 59.3 | 53.0 | 50.1-55.9 | 57.1 |
| 6. Age + | 66.0 | 63.3-68.7 | 70.8 | 60.4 | 57.6-63.2 | 64.6 | 66.9 | 64.1-69.5 | 71.5 | 65.6 | 62.8-68.3 | 68.2 |
| Morphokinetics | | | | | | | | | | | | |
| 7. Age + BS | 69.2 | 66.5-71.8 | 72.9 | 69.2 | 66.5-71.8 | 73.9 | 69.0 | 66.2-71.6 | 74.5 | 68.7 | 66.0-71.3 | 73.4 |
| 8. Age + BG | 69.4 | 66.7-72.0 | 73.3 | 66.3 | 63.5-69.0 | 70.1 | 68.7 | 66.0-71.3 | 74.0 | 64.8 | 62.1-67.6 | 73.9 |
| 9. Age + AIBS | 68.2 | 65.5-70.8 | 71.3 | 64.5 | 61.7-67.2 | 67.5 | 68.3 | 65.6-70.9 | 72.3 | 61.2 | 58.3-63.9 | 64.2 |
| 10. Age + | 70.0 | 67.3-72.6 | 74.5 | 66.4 | 63.7-69.1 | 72.1 | 69.3 | 66.6-71.9 | 75.6 | 69.0 | 66.3-71.7 | 71.1 |
| Morphokinetics + BS | | | | | | | | | | | | |
| 11. Age + | 69.6 | 66.9-72.2 | 73.2 | 63.3 | 60.5-66.1 | 67.3 | 68.7 | 66.0-71.3 | 72.7 | 68.9 | 66.2-71.5 | 72.0 |
| Morphokinetics + BG | | | | | | | | | | | | |
| 12. Age + | 67.5 | 64.8-70.2 | 70.5 | 63.3 | 60.4-66.0 | 64.8 | 68.3 | 65.6-70.9 | 70.6 | 67.9 | 65.1-70.5 | 69.8 |
| Morphokinetics + | | | | | | | | | | | | |
| AIBS | | | | | | | | | | | | |
| CA vs. EUP + SA | | | | | | | | | | | | |
| 1. Age | 73.1 | 70.5-75.6 | 48.4 | 73.1 | 70.5-75.6 | 48.4 | 73.1 | 70.5-75.6 | 48.4 | 73.1 | 70.5-75.6 | 48.4 |
| 2. Morphokinetics | 59.4 | 56.5-62.2 | 28.8 | 49.9 | 47.0-52.8 | 26.8 | 59.2 | 56.4-62.0 | 29.7 | 70.9 | 68.2-73.5 | 29.5 |
| 3. BS | 68.7 | 66.0-71.3 | 41.7 | . . . | . . . | . . . | 68.7 | 66.0-71.3 | 41.7 | 68.7 | 66.0-71.3 | 41.7 |
| 4. BG | 66.8 | 64.0-69.5 | 39.7 | 67.6 | 64.9-70.3 | 40.4 | 68.1 | 65.4-70.8 | 41.0 | 67.4 | 64.6-70.0 | 40.1 |
| 5. AIBS | 56.8 | 53.9-59.6 | 31.4 | 53.8 | 50.9-56.6 | 28.9 | 61.6 | 58.7-64.3 | 26.2 | 62.7 | 59.9-65.4 | 29.6 |
| 6. Age + | 71.5 | 68.8-74.0 | 45.8 | 62.6 | 59.8-65.3 | 36.5 | 69.9 | 67.2-72.5 | 44.4 | 76.4 | 73.9-78.8 | 55.7 |
| Morphokinetics | | | | | | | | | | | | |
| 7. Age + BS | 72.5 | 69.9-75.0 | 47.8 | 73.8 | 71.2-76.3 | 49.5 | 72.9 | 70.3-75.4 | 48.3 | 73.1 | 70.5-75.6 | 48.5 |
| 8. Age + BG | 72.4 | 69.8-74.9 | 47.6 | 68.5 | 65.7-71.1 | 43.0 | 71.3 | 68.6-73.9 | 46.2 | 68.5 | 65.7-71.1 | 42.7 |
| 9. Age + AIBS | 72.0 | 69.3-74.5 | 46.9 | 67.2 | 64.5-69.9 | 41.9 | 73.0 | 70.4-75.5 | 48.4 | 71.6 | 69.0-74.2 | 45.5 |
| 10. Age + | 73.7 | 71.1-76.1 | 49.2 | 66.6 | 63.9-69.3 | 40.9 | 73.2 | 70.6-75.7 | 48.7 | 78.2 | 75.7-80.5 | 61.0 |
| Morphokinetics + BS | | | | | | | | | | | | |
| 11. Age + | 74.0 | 71.4-76.5 | 49.7 | 63.4 | 60.6-66.2 | 37.6 | 72.4 | 69.8-74.9 | 47.6 | 76.8 | 74.3-79.1 | 56.2 |
| Morphokinetics + BG | | | | | | | | | | | | |
| 12. Age + | 73.5 | 70.9-76.0 | 48.9 | 63.8 | 61.0-66.6 | 38.2 | 71.8 | 69.2-74.4 | 46.9 | 77.6 | 75.1-79.9 | 58.7 |
| Morphokinetics + | | | | | | | | | | | | |
| AIBS | | | | | | | | | | | | |
| CA vs. EUP | | | | | | | | | | | | |
| 1. Age | 76.5 | 73.5-79.3 | 66.0 | 76.5 | 73.5-79.3 | 66.0 | 76.5 | 73.5-79.3 | 66.0 | 76.5 | 73.5-79.3 | 66.0 |
| 2. Morphokinetics | 59.1 | 55.7-62.4 | 44.1 | 53.8 | 50.4-57.2 | 38.8 | 56.3 | 52.9-59.6 | 40.9 | 59.9 | 56.5-63.2 | 42.4 |
| 3. BS | 71.0 | 67.8-74.0 | 60.9 | 71.0 | 67.8-74.0 | 60.9 | 71.0 | 67.8-74.0 | 60.9 | 71.0 | 67.8-74.0 | 60.9 |
| 4. BG | 68.7 | 65.4-71.8 | 55.7 | 68.5 | 65.3-71.6 | 55.6 | 69.5 | 66.3-72.5 | 57.1 | 70.4 | 67.2-73.5 | 59.5 |
| 5. AIBS | 59.4 | 56.0-62.7 | 45.4 | 57.7 | 54.3-61.0 | 43.1 | 61.9 | 58.5-65.1 | 47.8 | 59.1 | 55.7-62.4 | 43.2 |
| 6. Age + | 75.2 | 72.2-78.1 | 65.4 | 68.5 | 65.3-71.6 | 55.6 | 75.0 | 71.9-77.8 | 63.7 | 77.4 | 74.5-80.2 | 71.2 |
| Morphokinetics | | | | | | | | | | | | |
| 7. Age + BS | 77.7 | 74.7-80.4 | 66.8 | 76.6 | 73.6-79.4 | 66.0 | 77.7 | 74.7-80.4 | 68.3 | 76.8 | 73.9-79.6 | 66.1 |
| 8. Age + BG | 77.7 | 74.7-80.4 | 67.5 | 75.9 | 72.9-78.7 | 63.9 | 76.1 | 73.1-79.0 | 64.4 | 76.8 | 73.9-79.6 | 66.2 |
| 9. Age + AIBS | 75.9 | 72.9-78.7 | 65.5 | 74.2 | 71.1-77.1 | 62.1 | 77.7 | 74.7-80.4 | 67.8 | 69.9 | 66.7-73.0 | 58.0 |
| 10. Age + | 77.9 | 75.0-80.6 | 68.5 | 75.4 | 72.4-78.3 | 64.2 | 78.7 | 75.8-81.4 | 69.6 | 78.9 | 76.1-81.6 | 72.4 |
| Morphokinetics + BS | | | | | | | | | | | | |
| 11. Age + | 78.0 | 75.1-80.7 | 69.2 | 70.8 | 67.6-73.8 | 58.1 | 77.8 | 74.8-80.5 | 66.9 | 80.6 | 77.8-83.2 | 75.5 |
| Morphokinetics + BG | | | | | | | | | | | | |
| 12. Age + | 77.0 | 74.0-79.7 | 67.3 | 71.6 | 68.4-74.6 | 59.0 | 76.8 | 73.9-79.6 | 66.7 | 78.9 | 76.1-81.6 | 73.4 |
| Morphokinetics + | | | | | | | | | | | | |
| AIBS | | | | | | | | | | | | |

TABLE 11
Deep learning performance ANU vs EUP (samples with complete data only*).

| | Performance | | | | Stratified test data | | | | | |
|---|---|---|---|---|---|---|---|---|---|---|
| | Model | | | | | 35 < | 37 < | | | |
| | Accuracy | 95% | PPV | NPV | Age ≤ | Age ≤ | Age ≤ | Age > | Day | Day |
| | % | CI | % | % | 35 | 37 | 39 | 39 | 5 | 6 |
| ANU vs. EUP | | | | | | | | | | |
| 1. Image | 57.0 | 54.1-59.8 | 57.9 | 54.7 | 47.4 | 53.3 | 58.1 | 71.3 | 53.5 | 59.9 |
| 2. Image + | 56.5 | 53.7-59.4 | 59.2 | 52.9 | 53.7 | 53.3 | 54.5 | 65.2 | 53.5 | 59.2 |
| Morphokinetics | | | | | | | | | | |
| 3. Image + BG | 57.4 | 54.6-60.2 | 59.0 | 54.5 | 51.8 | 52.1 | 56.5 | 70.2 | 52.2 | 61.8 |
| 4. Image + BS | 61.4 | 58.6-644 | 63.7 | 58.4 | 51.0 | 57.4 | 60.5 | 66.3 | 54.2 | 67.5 |
| 5. Image + AIBS | 58.7 | 55.9-61.5 | 59.0 | 58.1 | 49.6 | 55.8 | 59.8 | 72.0 | 54.9 | 62.0 |
| 6. Image + Age | 67.8 | 65.0-70.4 | 71.1 | 64.2 | 70.0 | 52.9 | 63.1 | 82.6 | 65.0 | 70.2 |
| 7. Image + Age + | 66.3 | 63.5-69.0 | 69.1 | 63.1 | 68.1 | 55.8 | 58.5 | 81.2 | 63.3 | 68.8 |
| Morphokinetics | | | | | | | | | | |
| 8. Image + Age + BG | 68.9 | 66.2-71.5 | 73.5 | 64.4 | 71.7 | 57.9 | 63.1 | 80.9 | 64.6 | 72.5 |
| 9. Image + Age + BS | 69.2 | 66.5-71.8 | 72.3 | 65.8 | 73.0 | 57.9 | 61.8 | 81.9 | 65.0 | 72.8 |
| 10. Image + Age + AIBS | 68.0 | 65.2-70.6 | 70.5 | 65.0 | 70.3 | 54.1 | 62.5 | 62.6 | 64.8 | 70.7 |
| 11. Image + Age + BG + | 69.4 | 66.7-72.0 | 71.5 | 66.9 | 72.8 | 59.9 | 61.8 | 81.2 | 62.8 | 75.0 |
| Morphokinetics | | | | | | | | | | |
| 12. Image + Age + BS + | 69.0 | 66.3-71.7 | 69.2 | 68.9 | 73.6 | 57.9 | 60.1 | 82.3 | 64.2 | 73.1 |
| Morphokinetics | | | | | | | | | | |
| 13. Image + Age + AIBS + | 67.8 | 65.0-70.4 | 69.7 | 65.3 | 70.8 | 57.4 | 58.5 | 82.6 | 64.4 | 70.7 |
| Morphokinetics | | | | | | | | | | |
| CA vs. EUP + SA | | | | | | | | | | |
| 1. Image | 71.5 | 68.8-74.0 | 27.8 | 74.3 | 88.1 | 77.1 | 68.9 | 48.3 | 81.1 | 62.4 |
| 2. Image + | 65.5 | 62.7-68.2 | 34.5 | 77.5 | 75.3 | 62.2 | 66.1 | 55.4 | 75.6 | 56.0 |
| Morphokinetics | | | | | | | | | | |
| 3. Image + BG | 70.6 | 68.0-73.2 | 43.2 | 80.3 | 77.2 | 75.1 | 68.6 | 60.5 | 78.3 | 63.3 |
| 4. Image + BS | 69.5 | 66.8-72.2 | 42.5 | 81.6 | 71.8 | 71.9 | 70.0 | 64.3 | 76.9 | 62.5 |
| 5. Image + AIBS | 61.6 | 58.7-64.3 | 31.4 | 77.0 | 66.7 | 59.8 | 64.3 | 54.1 | 71.3 | 52.4 |
| 6. Image + Age | 71.0 | 68.3-73.5 | 45.8 | 86.4 | 91.6 | 72.3 | 56.1 | 58.2 | 74.0 | 68.1 |
| 7. Image + Age + | 74.2 | 71.7-76.7 | 50.2 | 83.2 | 91.3 | 80.7 | 63.2 | 57.8 | 79.5 | 69.2 |
| Morphokinetics | | | | | | | | | | |
| 8. Image + Age + BG | 76.4 | 73.9-78.8 | 54.5 | 83.7 | 90.8 | 79.9 | 67.9 | 63.6 | 80.4 | 72.7 |
| 9. Image + Age + BS | 77.2 | 74.7-79.5 | 55.6 | 85.2 | 91.3 | 81.5 | 69.3 | 63.3 | 81.6 | 73.0 |
| 10. Image + Age + AIBS | 75.8 | 73.3-78.2 | 53.7 | 82.3 | 91.9 | 79.9 | 67.5 | 60.2 | 82.8 | 69.2 |
| 11. Image + Age + BG + | 74.7 | 72.1-77.1 | 50.8 | 85.2 | 91.1 | 80.7 | 62.1 | 60.9 | 76.8 | 72.7 |
| Morphokinetics | | | | | | | | | | |
| 12. Image + Age + BS + | 76.8 | 74.3-79.1 | 54.9 | 84.7 | 89.7 | 79.1 | 69.3 | 65.6 | 81.9 | 71.8 |
| Morphokinetics | | | | | | | | | | |
| 13. Image + Age + AIBS + | 75.0 | 72.4-77.4 | 51.7 | 82.8 | 91.1 | 81.1 | 64.4 | 59.5 | 76.4 | 73.6 |
| Morphokinetics | | | | | | | | | | |
| CA vs. EUP | | | | | | | | | | |
| 1. Image | 61.4 | 58.0-64.7 | 46.4 | 69.6 | 67.2 | 63.0 | 57.1 | 55.4 | 74.1 | 49.9 |
| 2. Image + | 64.3 | 61.0-67.5 | 50.4 | 74.1 | 71.2 | 58.8 | 63.8 | 59.0 | 73.4 | 56.1 |
| Morphokinetics | | | | | | | | | | |
| 3. Image + BG | 66.8 | 63.5-69.9 | 53.8 | 74.2 | 72.2 | 64.2 | 64.8 | 62.6 | 70.4 | 63.5 |
| 4. Image + BS | 68.9 | 65.7-72.0 | 57.6 | 74.3 | 74.9 | 70.9 | 66.3 | 60.5 | 72.4 | 65.7 |
| 5. Image + AIBS | 63.9 | 60.5-67.1 | 49.8 | 70.2 | 71.6 | 67.3 | 62.2 | 50.8 | 74.6 | 54.1 |
| 6. Image + Age | 75.7 | 72.7-78.5 | 64.9 | 82.7 | 88.6 | 69.7 | 59.2 | 77.4 | 80.5 | 71.3 |
| 7. Image + Age + | 75.4 | 72.4-78.3 | 64.2 | 83.0 | 88.0 | 69.1 | 58.2 | 79.0 | 77.6 | 73.5 |
| Morphokinetics | | | | | | | | | | |
| 8. Image + Age + BG | 76.4 | 73.4-79.2 | 65.5 | 83.6 | 84.6 | 69.1 | 65.8 | 80.5 | 79.1 | 73.9 |
| 9. Image + Age + BS | 77.9 | 75.0-80.6 | 70.4 | 83.6 | 88.6 | 72.7 | 66.3 | 77.4 | 79.8 | 76.2 |
| 10. Image + Age + AIBS | 75.8 | 72.8-78.6 | 64.5 | 83.6 | 88.6 | 67.9 | 58.2 | 80.5 | 77.1 | 74.6 |
| 11. Image + Age + BG + | 78.7 | 75.8-81.4 | 71.6 | 82.4 | 87.6 | 73.3 | 68.4 | 80.0 | 80.5 | 77.1 |
| Morphokinetics | | | | | | | | | | |
| 12. Image + Age + BS + | 78.6 | 75.7-81.3 | 67.5 | 86.5 | 88.0 | 72.1 | 64.8 | 83.6 | 80.8 | 76.6 |
| Morphokinetics | | | | | | | | | | |
| 13. Image + Age + AIBS + | 77.1 | 74.1-79.9 | 66.2 | 84.5 | 89.0 | 70.9 | 61.2 | 80.0 | 80.0 | 74.4 |
| Morphokinetics | | | | | | | | | | |

TABLE 12
STORK-A confusion matrix ANU vs. EUP - image, age, morphokinetics,
and BS (samples with complete data only*).

| | Ground Truth | Ground Truth | |
|---|---|---|---|
| | Aneuploid | Euploid | |
| Predicted | 466 | 186 | 652 |
| Aneuploid | | | |
| Predicted | 179 | 361 | 540 |
| Euploid | | | |
| | 645 | 547 | 1192 |

| | Single | Complex | |
|---|---|---|---|
| | Aneuploid | Aneuploid | Euploid |
| | (SA) | (CA) | (EUP) |
| Total | 323 | 313 | 547 |
| Samples | | | |
| Percent | 61.7% | 83.4% | 66.0% |
| Correct | | | |

TABLE 13
STORK-A confusion matrix CA vs. EUP + SA - image, age,
morphokinetics, and BG (samples with complete data only*).

| | | Sensitivity: 83.8% | |
|---|---|---|---|
| | Specificity: 58.1% | Ground Truth | |
| | Ground Truth | Euploid + | |
| | Complex | Single | |
| | Aneuploid | Aneuploid | Accuracy: 77.2% |
| Predicted | 179 | 143 | 322 |
| Complex | | | |
| Aneuploid | | | |
| Predicted | 129 | 741 | 870 |
| Euploid + | | | |
| Single | | | |
| Aneuploid | | | |
| | 308 | 884 | 1192 |
| | Single | Complex | |
| | Aneuploid | Aneuploid | Euploid |
| | (SA) | (CA) | (EUP) |
| Total | 337 | 308 | 547 |
| Samples | | | |
| Percent | 73.0% | 58.1% | 90.5% |
| Correct | | | |

TABLE 14
STORK-A confusion matrix CA vs. EUP - image, age, morphokinetics,
and BS (samples with complete data only*).

| | Sensitivity: 78.2% | | |
|---|---|---|---|
| | Ground Truth | Specificity: 78.8% | |
| | Complex | Ground Truth | |
| | Aneuploid | Euploid | Accuracy: 78.6% |
| Predicted | 241 | 116 | 357 |
| Complex | | | |
| Aneuploid | | | |
| Predicted | 67 | 431 | 498 |
| Euploid | | | |
| | 308 | 547 | 855 |

### Example 9

### STORK-A API for Clinical Use

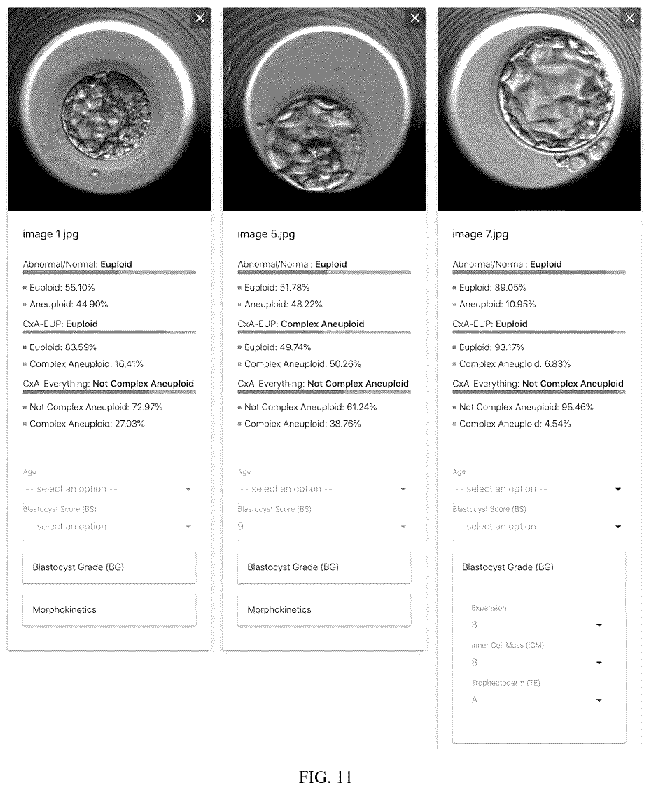

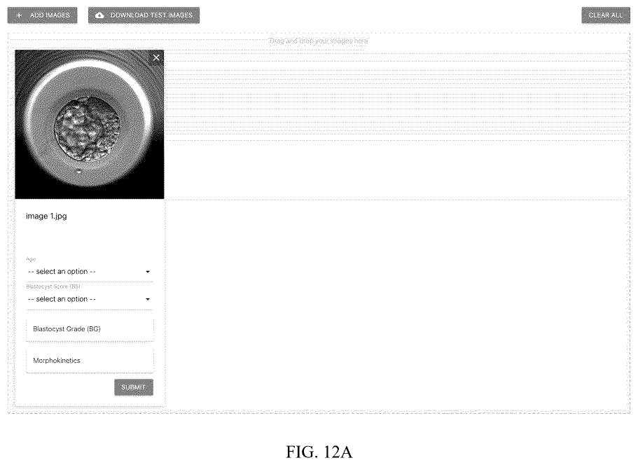

As part of this work, a web-based application programming interface (API) for STORK-A was developed (FIG. 11). The platform requires at a minimum an image of a blastocyst. Users then have the option to include patient age, morphological assessment, and complete morphokinetic parameters from tPnF to tSB. The results include probabilities for each of the three classifiers.

The STORK-A API is a user-friendly web interface that allows embryologists and clinicians to quickly predict embryo ploidy by utilizing images of embryos at 110 hpi, and inputting additional features, such as, for example, maternal age at the time of oocyte retrieval, morphological assessment (blastocyst score or blastocyst grade), and morphokinetic parameters (tPnF to tSB) as inputs. The morphological assessment, specifically the blastocyst grade (including inner cell mass, trophectoderm, and expansion grading), should be completely filled in to use morphological assessment as an input parameter. Similarly, the morphokinetic parameters should be completely filled in to be used as an input parameter. At a minimum, an image is required; the other inputs are optional.

After inputting the image and/or various clinical parameters, the interface reports probabilities for each of the following classification tasks: Aneuploid vs. Euploid, Complex Aneuploid vs. Euploid, and Complex Aneuploid vs. Not Complex Aneuploid (Euploid+Single Aneuploid). Single aneuploids exhibit one chromosomal abnormality, whereas complex aneuploids (CxA or CA) exhibit two or more chromosomal abnormalities. The back-end of the platform recognizes the inputs being included by the user and selects the appropriately trained model to be used. Each of the classification tasks has its own unique model weights to create predictions (probabilities).

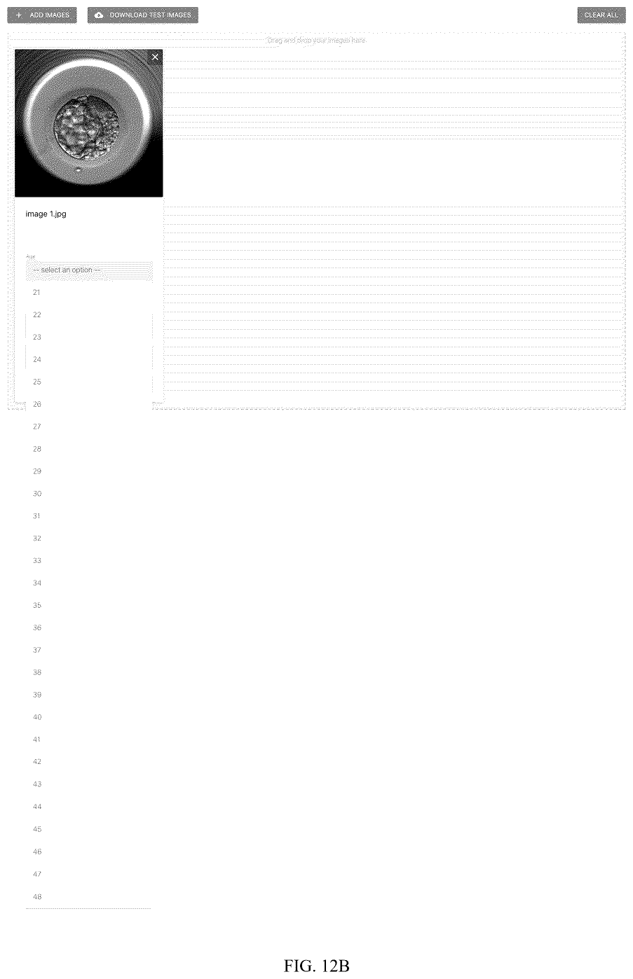

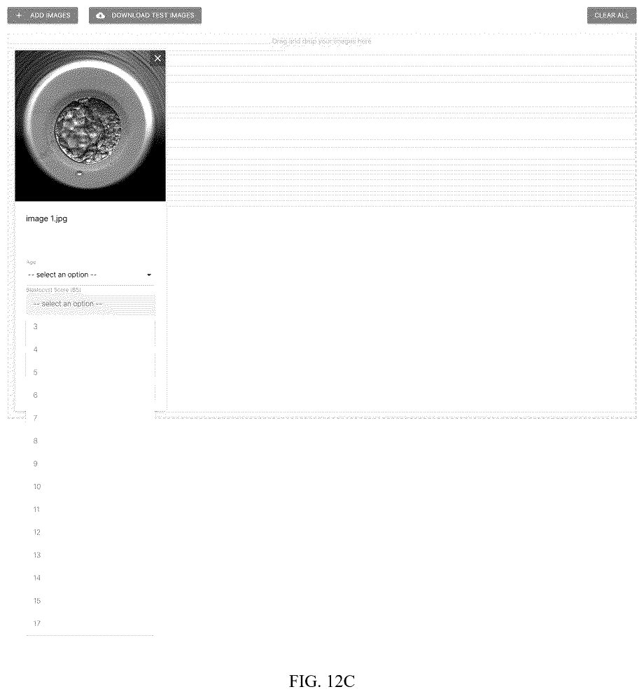

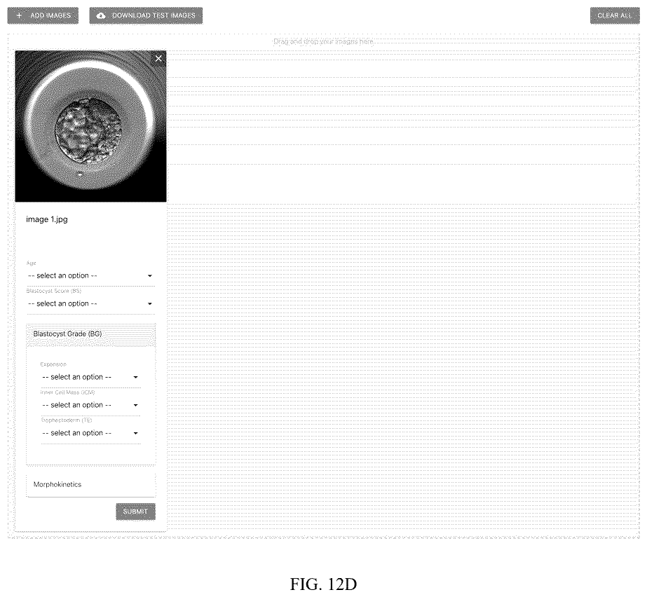

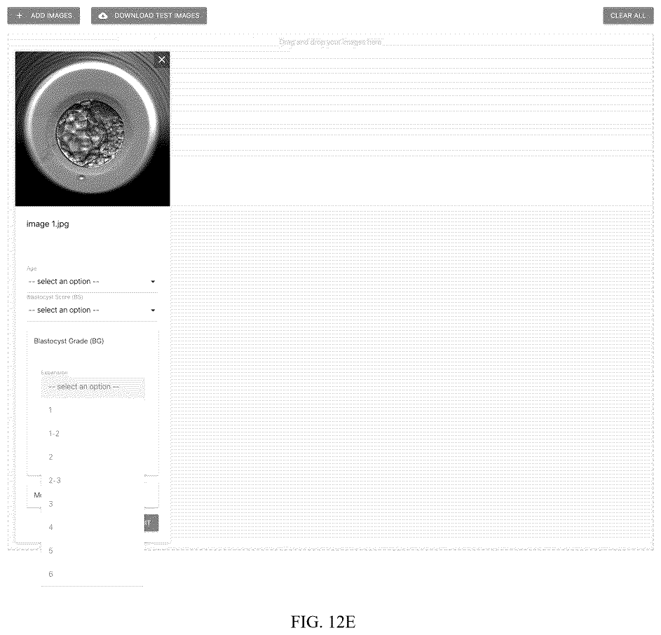

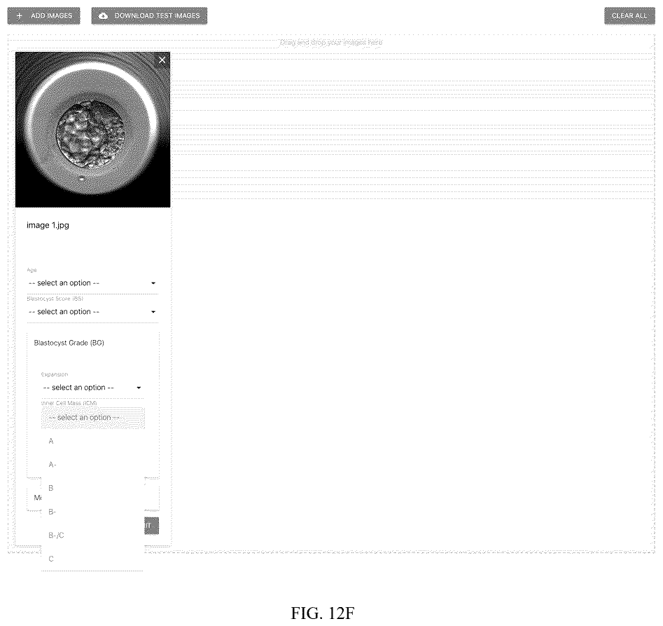

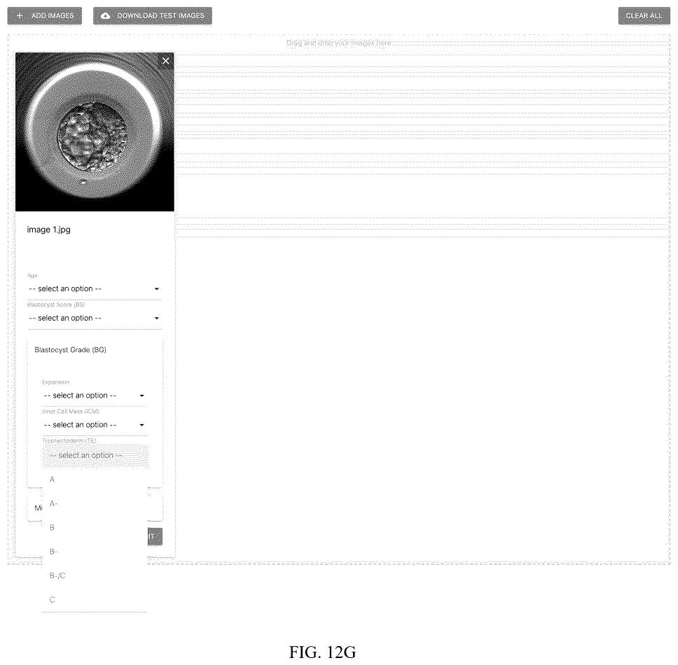

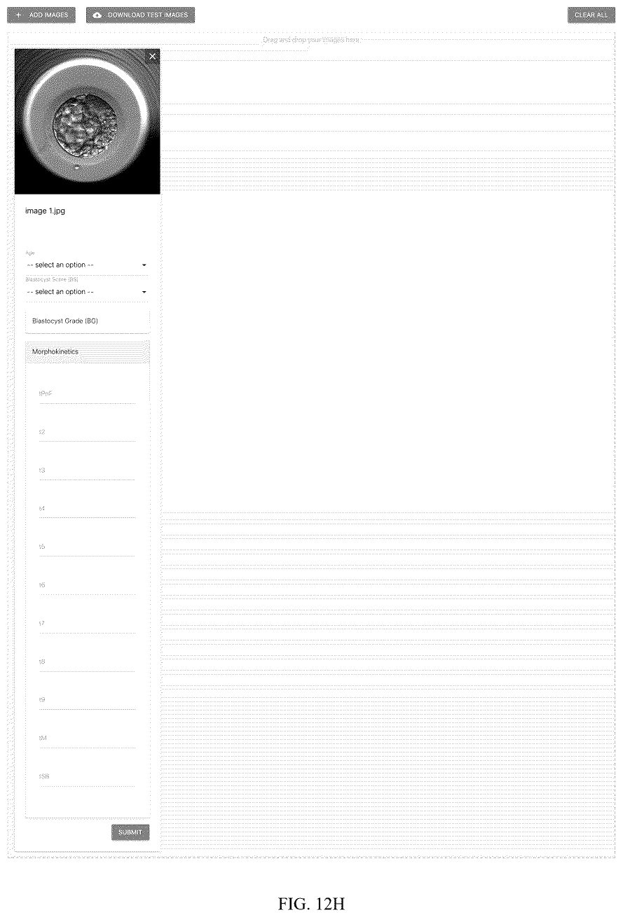

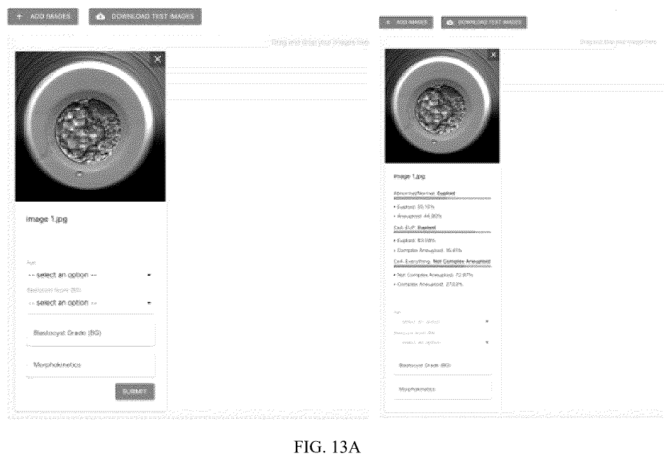

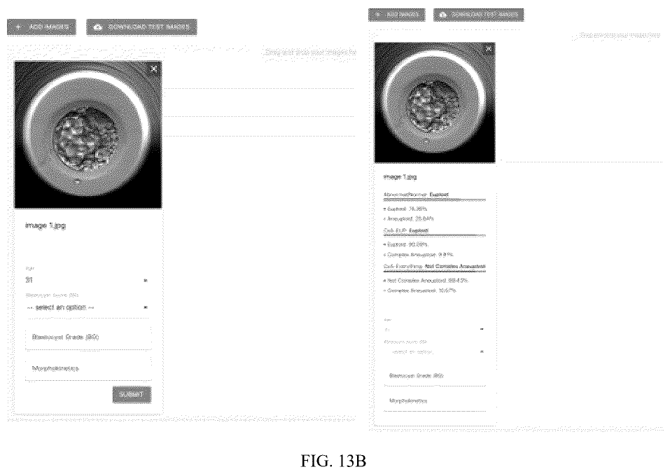

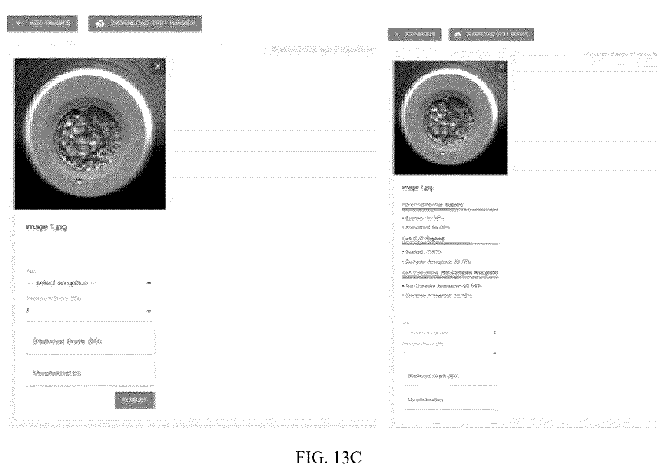

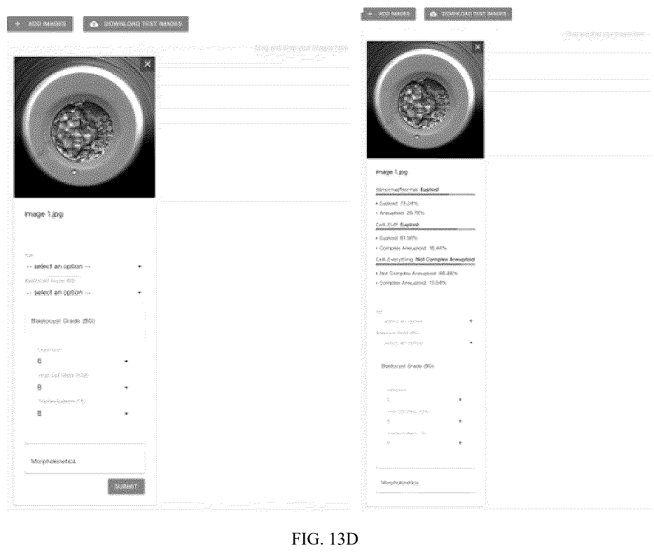

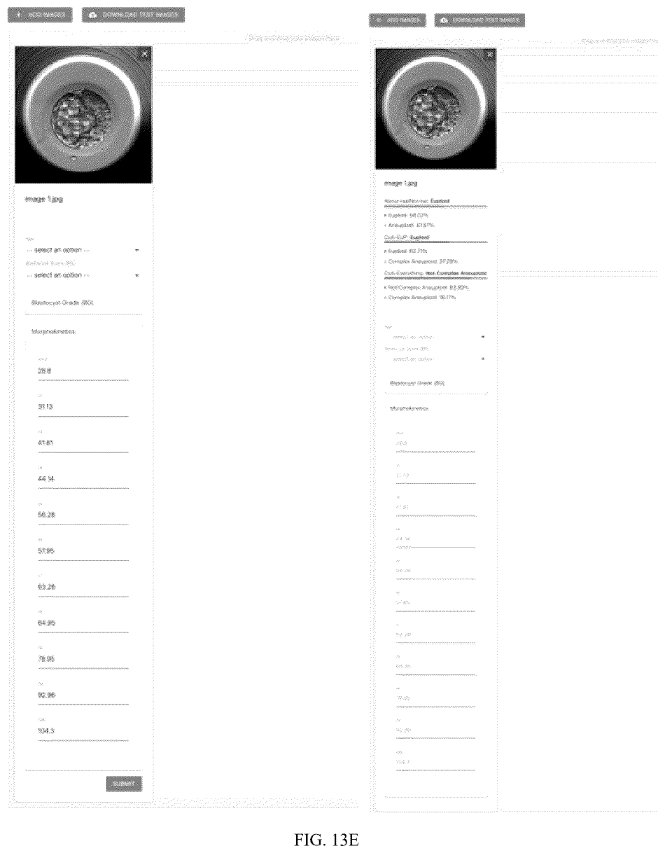

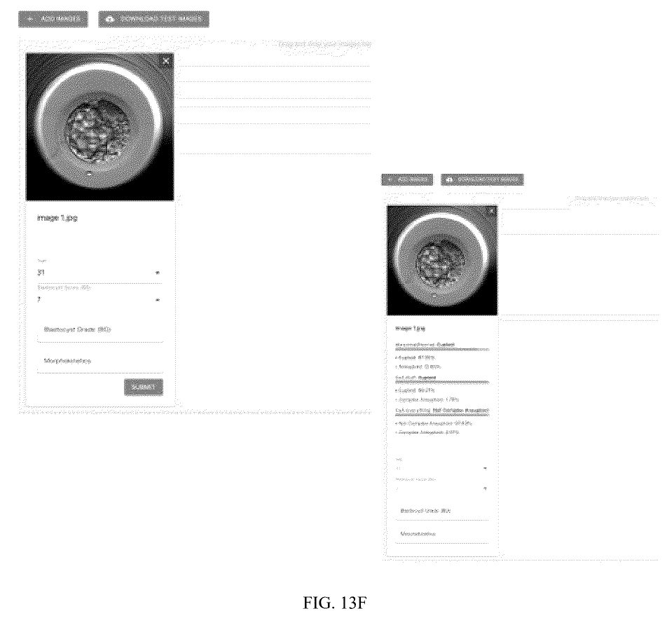

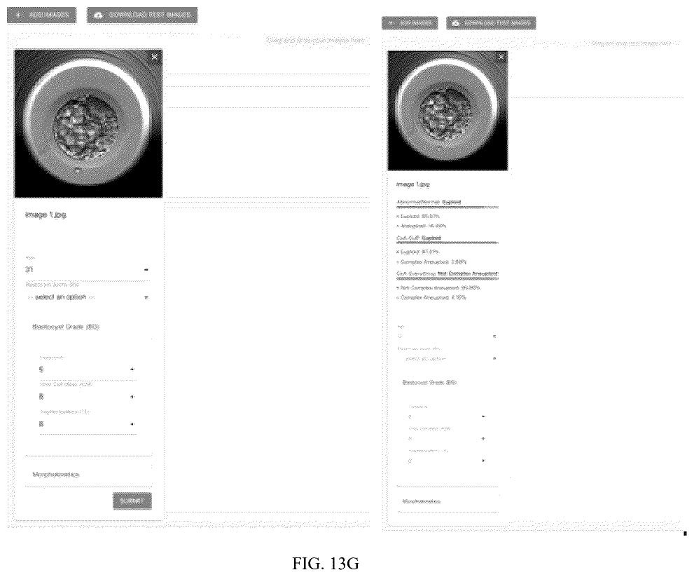

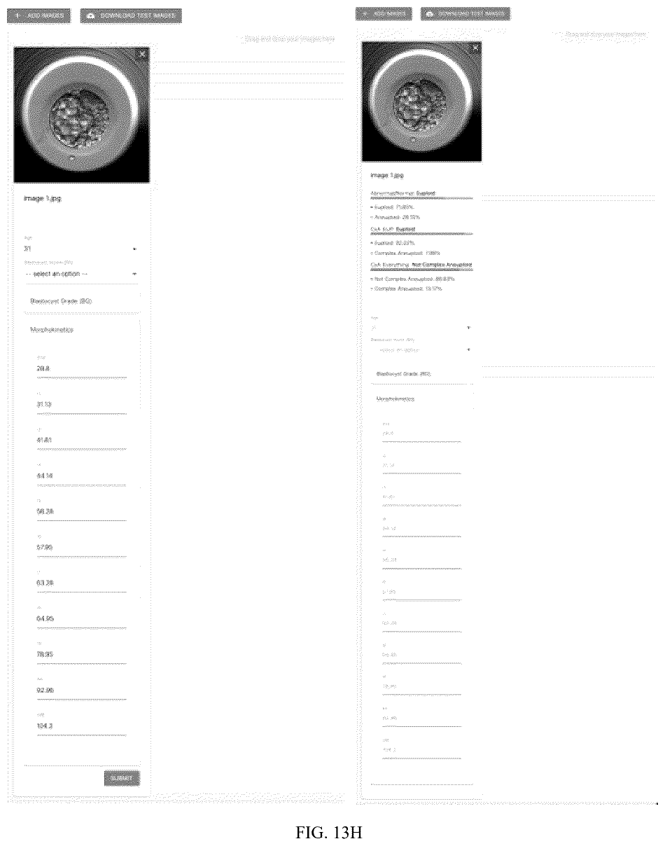

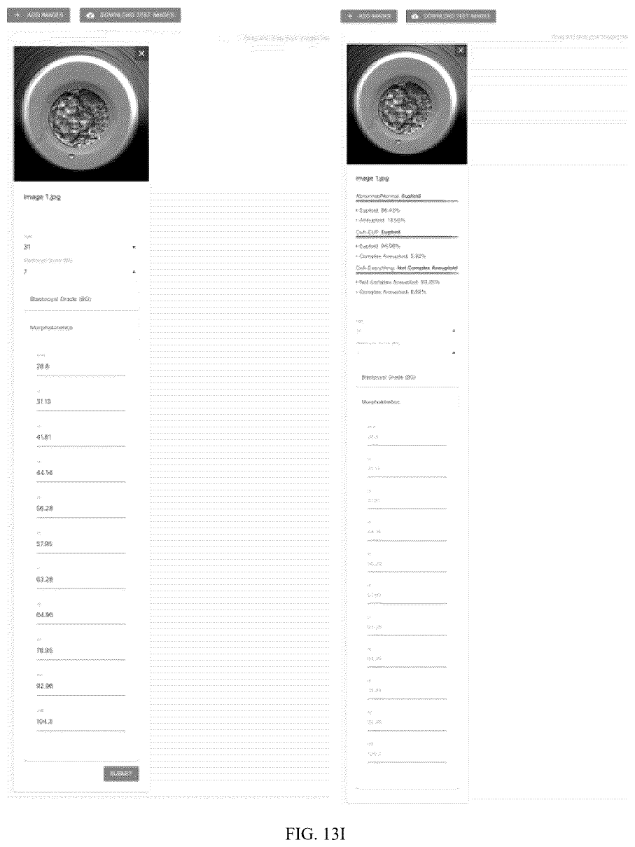

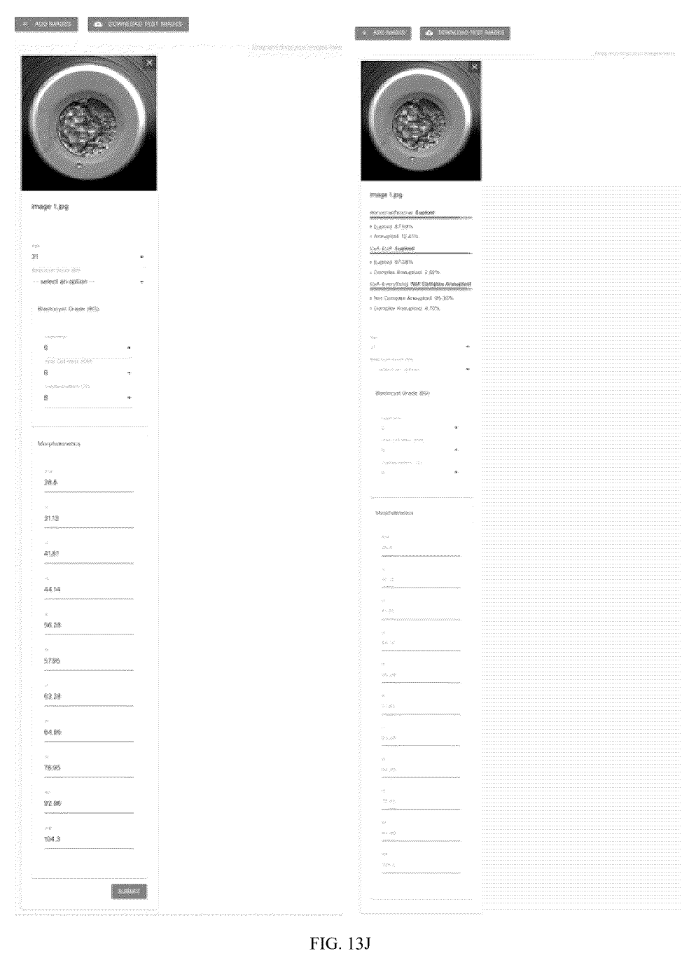

The API, showing the various STORK-A input parameters, is depicted in FIG. 12A-H. FIG. 13A-J depicts the influence of the various parameters, showing the STORK-A embryo ploidy prediction based on the image alone as compared to the embryo ploidy prediction based on the image in combination with one or more additional input parameters. The relative weights for each parameter input into the API are shown in Table 15 below.

TABLE 15
List of the various parameters input in the API in one example
of a prioritized order, and including definitions for each.

| Rank | Clinical Feature | Definition |
|---|---|---|
| 1. | Age | Patient age at the time of oocyte retrieval |
| 2. | Morphological Assessment | Assessment of the inner cell mass based on the Veeck |
| | (Blastocyst Grade - Inner | and Zaninovic** grading system. Grades included in |
| | cell mass) | API: A, A−, B, B−, B−/C, C. |
| | | A - tightly packed, compacted cells |
| | | B - larger, loose cells |
| | | C - ICM not readily distinguishable |
| | | D - cells of ICM appear degenerative |
| 3. | Morphological Assessment | Assessment of the trophectoderm based on the Veeck |
| | (Blastocyst Grade - | and Zaninovic grading system. Grades include in |
| | Trophectoderm) | API: A, A−, B, B−, B−/C, C. |
| | | A - healthy cells forming a cohesive epithelium |
| | | B - few, but healthy large cells |
| | | C - poor, very large, or unevenly distributed cells, |
| | | may appear as few cells squeezed to the side |
| | | D - cells of trophectoderm appear degenerative |
| 4. | Morphological Assessment | Assessment of the degree of expansion and hatching |
| | (Blastocyst Grade - | status based on the Veeck and Zaninovic grading |
| | Expansion) | system (27). Grades include in API: 1, 1-2, 2, 2-3, 3, |
| | | 4, 5, 6. |
| | | 1 - Early blastocyst; the blastocoel filling more than |
| | | half the volume of the conceptus, but no expansion in |
| | | overall size as compared to earlier stages |
| | | 2 - Blastocyst; the blastocoel filling more than half |
| | | of the volume of the conceptus; with slight expansion |
| | | in overall size and notable thinning of the zona |
| | | pellucida. |
| | | 3 - Full blastocyst; a blastocoel of more than 50% of |
| | | the conceptus volume and overall size fully enlarged |
| | | with a very thin zona pellucida |
| | | 4 - Hatching blastocyst; non-preimplantation genetic |
| | | diagnosis. The trophectoderm has started to herniate |
| | | through the zona |
| | | 5 - Fully hatched blastocyst; non-preimplantation |
| | | genetic diagnosis. Free blastocyst fully removed from |
| | | zona pellucida |
| | | 6 - Hatching or hatched blastocyst |
| 5. | Morphological Assessment | Blastocyst scores were derived from a system that |
| | (Blastocyst Score) | converts trophectoderm, inner cell mass, and |
| | | expansion grades from the Veeck and Zaninovic |
| | | grading system, into numerical values established by |
| | | Zhan et al. (28). Blastocyst score also takes into |
| | | consideration the day of blastocyst formation, i.e. |
| | | Day 5 vs. Day 6, when calculating the score. |
| 6. | Morphokinetic parameter | Time of pro-nuclear fading |
| | (tPnF) | |
| 7. | Morphokinetic parameter | Time to 2 cells |
| | (t2) | |
| 8. | Morphokinetic parameter | Time to 3 cells |
| | (t3) | |
| 9 | Morphokinetic parameter | Time to 4 cells |
| | (t4) | |
| 10. | Morphokinetic parameter | Time to 5 cells |
| | (t5) | |
| 11. | Morphokinetic parameter | Time to 6 cells |
| | (t6) | |
| 12. | Morphokinetic parameter | Time to 7 cells |
| | (t7) | |
| 13. | Morphokinetic parameter | Time to 8 cells |
| | (t8) | |
| 14. | Morphokinetic parameter | Time to 9 cells |
| | (t9) | |
| 15. | Morphokinetic parameter | Time of morula |
| | (tM) | |
| 16. | Morphokinetic parameter | Time of the start of blastulation |
| | (tSB) | |

### VIII. Additional Considerations

Any headers and/or subheaders between sections and subsections of this document are included solely for the purpose of improving readability and do not imply that features cannot be combined across sections and subsection. Accordingly, sections and subsections do not describe separate embodiments.

While the present teachings are described in conjunction with various embodiments, it is not intended that the present teachings be limited to such embodiments. On the contrary, the present teachings encompass various alternatives, modifications, and equivalents, as will be appreciated by those of skill in the art. The present description provides preferred exemplary embodiments, and is not intended to limit the scope, applicability, or configuration of the disclosure. Rather, the present description of the preferred exemplary embodiments will provide those skilled in the art with an enabling description for implementing various embodiments.

It is understood that various changes may be made in the function and arrangement of elements without departing from the spirit and scope as set forth in the appended claims. Thus, such modifications and variations are considered to be within the scope set forth in the appended claims. Further, the terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed.

In describing the various embodiments, the specification may have presented a method and/or process as a particular sequence of steps. However, to the extent that the method or process does not rely on the particular order of steps set forth herein, the method or process should not be limited to the particular sequence of steps described, and one skilled in the art can readily appreciate that the sequences may be varied and still remain within the spirit and scope of the various embodiments.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

Specific details are given in the present description to provide an understanding of the embodiments. However, it is understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

The various methods and techniques described above provide a number of ways to carry out the invention. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some preferred embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the invention extend beyond the specifically disclosed embodiments to other alternative embodiments and/or uses and modifications and equivalents thereof.

In some embodiments, the numbers expressing quantities of ingredients, properties such as molecular weight, reaction conditions, and so forth, used to describe and claim certain embodiments of the application are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

In some embodiments, the terms “a” and “an” and “the” and similar references used in the context of describing a particular embodiment of the application (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Preferred embodiments of this application are described herein. Variations on those preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In describing the various embodiments, the specification may have presented a method and/or process as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process should not be limited to the performance of their steps in the order written, and one skilled in the art can readily appreciate that the sequences may be varied and still remain within the spirit and scope of the various embodiments. Similarly, any of the various system embodiments may have been presented as a group of particular components. However, these systems should not be limited to the particular set of components, now their specific configuration, communication and physical orientation with respect to each other. One skilled in the art should readily appreciate that these components can have various configurations and physical orientations (e.g., wholly separate components, units and subunits of groups of components, different communication regimes between components).

In closing, it is to be understood that the embodiments of the application disclosed herein are illustrative of the principles of the embodiments of the invention. Although specific embodiments and applications of the disclosure have been described in this specification, these embodiments and applications are exemplary only, and many variations are possible. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

### REFERENCES

1 Herbert M, Kalleas D, Cooney D, Lamb M, Lister L. Meiosis and Maternal Aging: Insights from Aneuploid Oocytes and Trisomy Births. Cold Spring Harb Perspect Biol 2015; 7. DOI:101·101/cshperspect.a017970.
2 Gardner D K, Sakkas D. Assessment of Embryo Viability: The Ability to Select a Single Embryo for Transfer—a Review. Placenta 2003; 24: S5-12.
3 Gardner D K, Meseguer M, Rubio C, Treff N R. Diagnosis of human preimplantation embryo viability. Hum Reprod Update 2015; 21:727-47.
4 Meseguer M, Rubio I, Cruz M, Basile N, Marcos J, Requena A. Embryo incubation and selection in a time-lapse monitoring system improves pregnancy outcome compared with a standard incubator: a retrospective cohort study. Fertil Steril 2012; 98:1481-1489.e10.
5 Storr A, Venetis C A, Cooke S, Kilani S, Ledger W. Inter-observer and intra-observer agreement between embryologists during selection of a single Day 5 embryo for transfer: a multicenter study. Hum Reprod 2017; 32:307-14.
6 Tunis S R, Clarke M, Gorst S L, et al. Improving the relevance and consistency of outcomes in comparative effectiveness research. J Comp Eff Res 2016; 5:193-205.
7 Paternot G, Devroe J, Debrock S, D'Hooghe T M, Spiessens C. Intra- and inter-observer analysis in the morphological assessment of early-stage embryos. Reprod Biol Endocrinol 2009; 7:105.
8 Sundvall L, Ingerslev H J, Breth Knudsen U, Kirkegaard K. Inter- and intra-observer variability of time-lapse annotations. Human Reproduction 2013; 28:3215-21.
9 Khosravi P, Kazemi E, Zhan Q, et al. Deep learning enables robust assessment and selection of human blastocysts after in vitro fertilization. npj Digit Med 2019; 2:21.
10 Lee H L, McCulloh D H, Hodes-Wertz B, Adler A, McCaffrey C, Grifo J A. In vitro fertilization with preimplantation genetic screening improves implantation and live birth in women age 40 through
43. J Assist Reprod Genet 2015; 32:435-44.
11 Simon A L, Kiehl M, Fischer E, et al. Pregnancy outcomes from more than 1,800 in vitro fertilization cycles with the use of 24-chromosome single-nucleotide polymorphism-based preimplantation genetic testing for aneuploidy. Fertility and Sterility 2018; 110:113-21.
12 Munné S, Kaplan B, Frattarelli J L, et al. Preimplantation genetic testing for aneuploidy versus morphology as selection criteria for single frozen-thawed embryo transfer in good-prognosis patients: a multicenter randomized clinical trial. Fertility and Sterility 2019; 112:1071-1079.e7.
13 on behalf of ESHG, ESHRE and EuroGentest2, Harper J C, Geraedts J, et al. Current issues in medically assisted reproduction and genetics in Europe: research, clinical practice, ethics, legal issues and policy: European Society of Human Genetics and European Society of Human Reproduction and Embryology. Eur J Hum Genet 2013; 21: S1-21.
14 Xu J, Fang R, Chen L, et al. Noninvasive chromosome screening of human embryos by genome sequencing of embryo culture medium for in vitro fertilization. Proc Natl Acad Sci USA 2016; 113:11907-12.
15 Huang L, Bogale B, Tang Y, Lu S, Xie X S, Racowsky C. Noninvasive preimplantation genetic testing for aneuploidy in spent medium may be more reliable than trophectoderm biopsy. Proc Natl Acad Sci USA 2019; 116:14105-12.
16 Cimadomo D, Capalbo A, Ubaldi F M, et al. The Impact of Biopsy on Human Embryo Developmental Potential during Preimplantation Genetic Diagnosis. Biomed Res Int 2016; 2016. DOI: 101·155/2016/7193075.
17 Campbell A, Fishel S, Bowman N, Duffy S, Sedler M, Hickman C F L. Modelling a risk classification of aneuploidy in human embryos using non-invasive morphokinetics. Reproductive BioMedicine Online 2013; 26:477-85.
18 Basile N, Nogales M del C, Bronet F, et al. Increasing the probability of selecting chromosomally normal embryos by time-lapse morphokinetics analysis. Fertil Steril 2014; 101:699-704.
19 Kramer Y G, Kofinas J D, Melzer K, et al. Assessing morphokinetic parameters via time lapse microscopy (TLM) to predict euploidy: are aneuploidy risk classification models universal? J Assist Reprod Genet 2014; 31:1231-42.
20 Chawla M, Fakih M, Shunnar A, et al. Morphokinetic analysis of cleavage stage embryos and its relationship to aneuploidy in a retrospective time-lapse imaging study. J Assist Reprod Genet 2015; 32:69-75.
21 Minasi M G, Colasante A, Riccio T, et al. Correlation between aneuploidy, standard morphology evaluation and morphokinetic development in 1730 biopsied blastocysts: a consecutive case series study. Human Reproduction 2016; 31:2245-54.
22 Patel D, Shah P, Kotdawala A, Herrero J, Rubio I, Banker M. Morphokinetic behavior of euploid and aneuploid embryos analyzed by time-lapse in embryoscope. J Hum Reprod Sci 2016; 9:112.
23 Mumusoglu S, Yarali I, Bozdag G, et al. Time-lapse morphokinetic assessment has low to moderate ability to predict euploidy when patient—and ovarian stimulation-related factors are taken into account with the use of clustered data analysis. Fertility and Sterility 2017; 107:413-421.e4.
24 Del Carmen Nogales M, Bronet F, Basile N, et al. Type of chromosome abnormality affects embryo morphology dynamics. Fertil Steril 2017; 107:229-235.e2.
25 Chavez-Badiola A, Flores-Saiffe-Farias A, Mendizabal-Ruiz G, Drakeley A J, Cohen J. Embryo Ranking Intelligent Classification Algorithm (ERICA): artificial intelligence clinical assistant predicting embryo ploidy and implantation. Reproductive BioMedicine Online 2020; 41:585-93.
26 Lee C I, Su Y R, Chen C H, et al. End-to-end deep learning for recognition of ploidy status using time-lapse videos. J Assist Reprod Genet 2021; 38:1655-63.
27 An Atlas of Human Blastocysts. Routledge & CRC Press. https://www.routledge.com/An-Atlas-of-Human-Blastocysts/Veeck-Zaninovic/p/book/9780367395285 (accessed Mar. 12, 2021).
28 Zhan Q, Sierra E T, Malmsten J, Ye Z, Rosenwaks Z, Zaninovic N. Blastocyst score, a blastocyst quality ranking tool, is a predictor of blastocyst ploidy and implantation potential. F & S Reports 2020; 1:133-41.
29 Lundberg S, Lee S I. A Unified Approach to Interpreting Model Predictions. ar Xiv:170507874 [cs, stat] 2017; published online November 24. http://arxiv.org/abs/17050.7874 (accessed Mar. 12, 2021).
30 He K, Zhang X, Ren S, Sun J. Deep Residual Learning for Image Recognition. In: 2016 IEEE Conference on Computer Vision and Pattern Recognition (CVPR). 2016:770-8.
31 Irani M, Zaninovic N, Rosenwaks Z, Xu K. Does maternal age at retrieval influence the implantation potential of euploid blastocysts? American Journal of Obstetrics and Gynecology 2019; 220:379.e1-379.e7.
32 Demko Z P, Simon A L, McCoy R C, Petrov D A, Rabinowitz M. Effects of maternal age on euploidy rates in a large cohort of embryos analyzed with 24-chromosome single-nucleotide polymorphism-based preimplantation genetic screening. Fertility and Sterility 2016; 105:1307-13.
33 Chen T J, Zheng W L, Liu C H, Huang I, Lai H H, Liu M. Using Deep Learning with Large Dataset of Microscope Images to Develop an Automated Embryo Grading System. FandR 2019; 01:51-6.
34 Bormann C L, Kanakasabapathy M K, Thirumalaraju P, et al. Performance of a deep learning based neural network in the selection of human blastocysts for implantation. eLife 2020; 9: e55301.
35 Cornelisse S, Zagers M, Kostova E, Fleischer K, Wely M, Mastenbroek S. Preimplantation genetic testing for aneuploidies (abnormal number of chromosomes) in in vitro fertilisation. Cochrane Database Syst Rev 2020; 2020: CD005291.

1. A non-invasive method of predicting ploidy status of an embryo, the method comprising:
receiving a dataset comprising a static image of the embryo;
analyzing the dataset by one or more machine and/or deep learning model via one or more classification task applied to the dataset; and
generating an output prediction of the ploidy status of the embryo.

2. The method of claim 1, wherein the prediction of the ploidy status of the embryo comprises a probability of the embryo being euploid.

3. (canceled)

4. The method of claim 2, wherein the classification task is a binary classification task.

5. The method of claim 4, wherein the binary classification task provides a probability for the embryo of being aneuploid vs. euploid; complex aneuploid vs. euploid or single aneuploid; or complex aneuploid vs. euploid.

6-8. (canceled)

9. The method of claim 1, the method further comprising acquiring the static image; and/or wherein the static image is acquired via time-lapse microscopy, is captured at Day 5 or Day 6 of embryo development, is captured from 105-115, or from 109-111 hours post insemination (hpi), and/or is captured at or about 110 hours post insemination (hpi); and/or wherein one individual static image is captured and analyzed per embryo.

10-14. (canceled)

15. The method of claim 1, wherein the dataset further comprises one or more clinical and/or morphological features for the embryo, wherein the clinical and/or morphological features comprise one or more morphokinetic parameters/annotations, one or more blastocyst morphological assessments, maternal age at the time of oocyte retrieval, and/or preimplantation genetic testing for aneuploidy (PGT-A).

16. (canceled)

17. The method of claim 15, wherein:
the blastocyst morphological assessments comprise blastocyst grade (BG), blastocyst score (BS), and/or artificial intelligence-driven predicted blastocyst score (AIBS); and/or
the morphokinetic parameters comprise time of pro-nuclear fading (tPnF), time to 2 cells (t2), time to 3 cells (t3), time to 4 cells (t4), time to 5 cells (t5), time to 6 cells (t6), time to 7 cells (t7), time to 8 cells (18), time to 9 cells (t9), time of morula (tM), and/or time of the start of blastulation (tSB).

18. The method of claim 17, wherein;
the blastocyst score (BS) is determined based on machine and/or deep learning and regression analysis; and/or
the blastocyst score (BS) determination comprises converting inner cell mass (ICM), trophectoderm (TE), and/or expansion grades into numerical values, and additionally comprises an input based on day of blastocyst formation; and/or
analyzing morphokinetic parameters comprises assigning blastocyst grade (BG) using a grading system.

19-20. (canceled)

21. The method of claim 18, wherein blastocyst score (BS) further comprises a score based on day of blastocyst formation and/or the grading system comprises assessments of inner cell mass (ICM), trophectoderm (TE), and/or expansion.

22-24. (canceled)

25. The method of claim 15, wherein:
the clinical features comprise maternal age and/or blastocyst score (BS);
maternal age and/or blastocyst score (BS) are weighted more heavily than other clinical features based on one or more classification task;
trophectoderm (TE) score is weighted more heavily than other blastocyst score factors based on one or more classification tasks;
the clinical and/or morphological features comprise one or more of maternal age at the time of oocyte retrieval, blastocyst grade-inner cell mass, blastocyst grade-trophectoderm, blastocyst grade-expansion, blastocyst score, time of pro-nuclear fading (tPnF), time to 2 cells (t2), time to 3 cells (t3), time to 4 cells (t4), time to 5 cells (t5), time to 6 cells (t6), time to 7 cells (t7), time to 8 cells (t8), time to 9 cells (t9), time of morula (tM), and/or time of the start of blastulation (tSB); and/or
the clinical and/or morphological features are weighted in order of maternal age at the time of oocyte retrieval, blastocyst, blastocyst score, and/or morphokinetic parameters.

26-31. (canceled)

32. The method of claim 1, the method further comprising pre-processing the dataset prior to analysis.

33. The method of claim 32, wherein pre-processing the dataset comprises removing faulty static images and/or imputing values for any missing morphokinetic parameters via median imputation; and/or the dataset comprises values for each morphokinetic parameter following pre-processing.

34-35. (canceled)

36. The method of claim 1, wherein the analysis comprises regression analysis and/or determination of an artificial intelligence-driven predicted blastocyst score (AIBS) for the embryo.

37. The method of claim 36, wherein the regression analysis:
comprises a LASSO regression and/or logistic regression applied to one or more clinical features; and/or
is used to weight importance of one or more clinical features.

38-39. (canceled)

40. The method of claim 1, wherein the static image(s) and clinical features are combined and analyzed by machine and/or deep learning in two fully-connected layers and/or wherein the machine learning comprises a convolutional neural network (CNN), a ResNet18 CNN architecture, Extreme Gradient Boost Decision Tree (XGBoost), k-nearest neighbor (k-NN), support vector machine (SVM), and/or Random Forest.

41-44. (canceled)

45. The method of claim 1, the method further comprising:
(a) training the one or more machine learning model using training data, wherein the training data comprises a plurality of probabilities, and/or model- or embryologist-derived or provided clinical features for a plurality of subjects and a plurality of embryo ploidy statuses for the plurality of subjects; and/or
(b) predicting embryo viability based on the embryo ploidy status, wherein an embryo having a stronger probability of being euploid has a higher probability of being viable.

46. (canceled)

47. The method of claim 1, wherein:
the method is used for improving embryo selection for implantation during in vitro fertilization;
the method is used for selecting and/or prioritizing an embryo for preimplantation genetic testing for aneuploidy (PGT-A) biopsy and/or implantation during in vitro fertilization; and/or
the method is used in combination with traditional methods of embryo selection and prioritization for implantation and/or recommendation for PGT-A during in vitro fertilization.

48-49. (canceled)

50. A method of improving an outcome in a subject undergoing in vitro fertilization, comprising the method of claim 1, wherein an embryo predicted to be euploid is selected for embryo transfer during in vitro fertilization.

51. (canceled)

52. A system comprising:
one or more data processors; and
a non-transitory computer readable storage medium containing instructions which, when executed on the one or more data processors, cause the one or more data processors to perform part or all of the method of claim 1.

53. A computer-program product tangibly embodied in a non-transitory machine-readable storage medium, including instructions configured to cause one or more data processors to perform part or all of the method of claim 1.

54. A user interface for predicting ploidy status of an embryo, the user interface comprising:
a web-based platform for uploading and analyzing a dataset, wherein the dataset comprises a static image of the embryo;
analysis software integrated with the web-based platform to analyze the dataset by one or more machine and/or deep learning model via one or more classification task applied to the dataset; and
an output generation which provides a prediction of ploidy status of the embryo.

55-96. (canceled)
